(19) United States
(12) Patent Application Publication
(10) Pub. No.: US 2025/0063951 A1
(43) Pub. Date: Feb. 20, 2025

(54) NOVEL PIEZOELECTRIC TRANSITION METAL HALOMETALLATES

(71) Applicant: UNIVERSITY OF KENTUCKY RESEARCH FOUNDATION, Lexington, KY (US)

(72) Inventors: Aron Huckaba, Lexington, KY (US); Michael Wells, Lexington, KY (US); Jacob Hempel, Lexington, KY (US)

(73) Assignee: UNIVERSITY OF KENTUCKY RESEARCH FOUNDATION, Lexington, KY (US)

(21) Appl. No.: 18/712,573

(22) PCT Filed: Nov. 22, 2022
(86) PCT No.: PCT/US22/80339

§ 371 Date: May 22, 2024

Related U.S. Application Data
(60) Provisional application No. 63/283,061, filed on Nov. 24, 2021.

(51) Int. Cl. H10N 30/853; C07F 1/08; C07F 3/06; C07F 15/06; C08K 5/00

(52) U.S. Cl. CPC H10N 30/853; C07F 1/08; C07F 3/06; C07F 15/065; C08K 5/0091

(57) ABSTRACT

The development of lead-free high performing novel piezoelectric materials could allow for lightweight and non-toxic piezoelectric energy harvesters. Herein, is disclosed novel organic-inorganic hybrid metalates (OIHM) incorporating histammonium salts of HistammoniumMXₙ, with M being a metal and X being a halide. Composites with non-reactive polymer composites such as polyvinylidene difluoride (PVDF) displayed exhibit excellent flexibility and promising unpoled piezoelectric coefficients.

### CROSS-REFERENCE TO RELATED APPLICATIONS

This application claims priority to U.S. Provisional Patent Application 63/283,061, filed Nov. 24, 2021, the contents of which are hereby incorporated by reference in their entirety.

### GOVERNMENT SUPPORT

This invention was made with support from grant 1849213 from the National Science Foundation. The government may have rights to the invention.

### TECHNICAL FIELD

The present disclosure generally relates to organic-inorganic halometallates that function as piezoelectric materials both in single-crystalline and in porous composite films.

### BACKGROUND

In piezoelectric sensors and energy harvesters, strain, force, or pressure are converted into electric charge. Piezoelectric materials are solids that lack an inversion center and produce an electrical potential from the change in dipole-moment caused by mechanical stressors or vice versa, a mechanical deformation in response to electrical stimulation. ⁽¹⁻¹¹⁾ Research into novel piezoelectrics is motivated by the search for alternatives to ceramics like lead zirconium titanate (PZT), which exhibit strong piezoelectric behavior but are rigid, ceramic materials that are energy intensive to make, and in the case of PZT, contain hazardous Lead. Poly(vinylidene difluoride) (PVDF) is often used in place of ceramic piezoelectrics, due to its inert and flexible nature, however, it exhibits a much weaker piezoelectric response than PZT.

It would therefore be useful to find novel piezoelectrics that can be fabricated with less energy while providing a strong piezoelectric response. State-of-the-art approaches to developing better piezoelectric materials have focused on pairing singly charged ammonium cations with transition metals, which are solution processable, formed at low temperatures, and exhibit various properties that can be tailored through materials selection and synthesis conditions.

### SUMMARY

Provided in the present disclosure are novel hybrid organic-inorganic halometallates with a dication, histammonium dichloride (HistNH₃), which has two different cation moieties, one primary ammonium and one imidazolium cation.

The present disclosure provides for halometallate compositions of a histammonium metal halide. The histammonium metal halide may be of the formula HistNH₃MXₙ, wherein M is a metal and X is a halide ion. The metal may be selected from iron (Fe), copper (Cu), cobalt (Co), nickel (Ni), zinc (Zn), antimony (Sb), tin (Sn), cadmium (Cd), mercury (Hg), lead (Pb), bismuth (Bi), scandium (Sc), titanium (Ti), vanadium (V), chromium (Cr), manganese (Mn), gallium (Ga), germanium (Ge), and arsenic (As), zirconium (Zr), molybdenum (Mo), ruthenium (Ru), rhodium (Rh), palladium (Pd), silver (Ag), and gold (Au). The halide ion, X, may be selected from chlorine (Cl), fluorine (F), bromine (Br), Iodine (I), and astatine (At).

In some aspects, the histammonium metal halide is selected from HistNH₃CoCl₄, HistNH₃CuCl₄, HistNH₃ZnCl₄, HistNH₃NiCl₄, HistNH₃CdCl₄, HistNH₃HgCl₄, HistNH₃SnCl₄, HistNH₃PbCl₄, HistNH₃SbCl₅, (HistNH₃)₃(BiCl₆)₂, HistNH₃PbBr₄, HistNH₃HgBr₄, HistNH₃SnBr₄, HistNH₃MnCl₄, HistNH₃PdCl₄, HistNH₃PdBr₄, HistNH₃RuCl₄, HistNH₃RuBr₄, HistNH₃ZnBr₄, HistNH₃MnBr₄, HistNH₃CoBr₄, HistNH₃NiBr₄, HistNH₃CuBr₄, (HistNH₃)₃(BiBr₆)₂, HistNH₃RuI₄, HistNH₃ZnI₄, HistNH₃MnI₄, HistNH₃CoI₄, HistNH₃NiI₄, HistNH₃CuI₄, (HistNH₃)₃(BiI₆)₂, HistNH₃PdI₄, HistNH₃PbI₄, HistNH₃HgI₄, HistNH₃SnI₄, HistNH₃SbI₅, and HistNH₃CdI₄.

The composition may further include a non-reactive polymer composite. The polymer composite may in some aspects be selected from polyvinylidene fluoride (PVDF), polydimethylsiloxane (PDMS), polydiethylsiloxane (PDES), polymethylphenylsiloxane (PMPS), polyvinylidene chloride (PVDC), cellulose acetate, ethylene vinyl acetate, high density polyethylene, polycarbonate, polyester (PET or PEN), polylactic acid, polyurethane, polyvinyl alcohol, polyvinyl butyral, polyvinyl chloride, polyvinyl fluoride, polypropylene, polystyrene, low density polyethylene, cellulose, polytetrafluoroethylene (PTFE), polyimide, melamine, nylon, or combinations thereof. In some aspects, the polymer composite is PVDF or PDMS.

The present disclosure further provides a piezoelectric device that includes the halometallate compositions disclosed herein. In some aspects, the piezoelectric device further includes a first electrode in contact with the halometallate composition. In some aspectrs, the piezoelectric device may also include a substrate, such as a glass or polymer. In some aspects, the piezoelectric may include a second electrode in contact with the halometallate composition. In some aspects, a first surface of the halometallate composition is layered at least in part on the first electrode and a second surface of the halometaalate composition is layered at least in part on the second electrode. In some aspects, the substrate is also layered on the first electrode.

### BRIEF DESCRIPTION OF THE DRAWINGS

FIG. 1 shows a typical load-displacement curve in a nanoindentation experiment.

FIG. 2 shows (a) Crystal structure of the hybrid organic-inorganic histammonium chlorozincate (HistNH₃ZnCl₄) in this study. (b) An example of a standard load-displacement curve on the Zn sample. Inset shows an optical image of the indent from this load-displacement curve. Radial cracks can be seen optically. The scale bar is 10 mm.

FIG. 3 shows the location of HistNH₃ZnCl₄ on the plot relating H/E to the ratio of irreversible work to total work.

FIG. 4 shows plots of all the nanoindentation curves taken on HistNH₃ZnCl₄.

FIG. 5 shows an optical image of all 16 indents made on HistNH₃ZnCl₄. The indents at the top right were taken after this set of 16 indents.

FIG. 6 shows optical images and measurements of the crack length for 6 indents. Each indent produced 2 cracks, thus giving 12 data points.

FIG. 7 shows crystallographically determined X-ray structures of (a) HistNH₃FeCl₄ and (b) HistNH₃(FeCl₄)₂.

FIG. 8 shows crystallographically determined X-ray structures of (a) HistNH₃CoCl₄ and (b) HistNH₃ZnCl₄.

FIG. 9 shows crystallographically determined X-ray structures of (a) [HistNH₃Ni(H₂O)Cl₃]Cl and (b) HistNH₃CuCl₄.

FIG. 10 shows crystallographically determined X-ray structures of (a) HistNH₃CdCl₄ and (b) HistNH₃HgCl₄.

FIG. 11 shows crystallographically determined X-ray structures of (a) HistNH₃SnCl₄, (b) HistNH₃PbCl₄, and (c) HistNH₃SbCl₄.

FIG. 12 shows crystallographically determined X-ray structure of (HistNH₃)₃(BiCl₆)₂.

FIG. 13 shows elementwise contribution on the density of states (DOS) per electrons per atom for (a) HistNH₃ZnCl₄, (b) HistNH₃CoCl₄, (c) HistNH₃CuCl₄, and (d) HistNH₃SbCl₅ and projected crystal orbital Hamilton population (pCOHP) of cation-Cl bond in (e) HistNH₃ZnCl₄, (f) HistNH₃CoCl₄, (g) HistNH₃CuCl₄, and (h) HistNH₃SbCl₅.

FIG. 14 shows the average local piezoelectric responses for all three polymer composite samples without poling, compared with a reference PVDF polymer sample and a single-crystal HistNH₃ZnCl₄ sample. Above each sample is a 60×60 μm topography image of each material, taken using contact mode at a scan rate of 0.1 Hz, with a scale bar of 20 μm. Listed from left to right are the HistNH₃ZnCl₄ (single crystal), PVDF reference film, HistNH₃ZnCl₄·PVDF composite, HistNH₃CoCl₄·PVDF composite and HistNH₃CuCl₄·PVDF composite.

FIG. 15 shows Second Harmonic Generation activity for single crystals of HistNH₃ZnCl₄ (circles), HistNH₃CoCl₄ (squares), and HistNH₃SbCl₅ (triangles), a sheet of poled PVDF (diamonds), and single crystals of KDP (crosses). The incidence of light has been normalized for each of the samples.

FIG. 16 shows various arrangements for piezoelectric devices.

### DESCRIPTION

The present disclosure provides for novel hybrid organic-inorganic halometallates, referred to as halometallates herein. Such may also be referred to as hybrid organic inorganic metallates (OIHMs). The halometallate compounds include a dication, histammonium dichloride (HistNH₃), which has two different cation moieties, one primary ammonium and one imidazolium cation. Because the two cations have different steric profiles and vastly different hydrogen bonding patterns, major differences in cation placement within the crystalline framework was expected.

### Piezoelectric Compositions

The compositions of the present disclosure include the base compound HistNH₃MX, wherein X is a halide and M is selected from iron (Fe), copper (Cu), cobalt (Co), nickel (Ni), zinc (Zn), antimony (Sb), tin (Sn), cadmium (Cd), mercury (Hg), lead (Pb), bismuth (Bi), scandium (Sc), titanium (Ti), vanadium (V), chromium (Cr), manganese (Mn), gallium (Ga), germanium (Ge), and arsenic (As), zirconium (Zr), molybdenum (Mo), ruthenium (Ru), rhodium (Rh), palladium (Pd), silver (Ag), and gold (Au). In some aspects, M may include iron (Fe), copper (Cu), cobalt (Co), nickel (Ni), zinc (Zn), antimony (Sb), tin (Sn), cadmium (Cd), mercury (Hg), lead (Pb), and bismuth (Bi). In some aspects, M is a 3d metal. It will be understood that X may include chlorine (Cl), fluorine (F), bromine (Br), Iodine (I), and astatine (At). In some aspects, the halometallates may be of metal halides M^{x+}Clₓ, where M=Fe²⁺, Fe³⁺, Cu²⁺, Co²⁺, Ni²⁺, Zn²⁺, Cd²⁺, Hg²⁺, Sn²⁺, Pb²⁺, Sb²⁺, or Bi³⁺. The compounds herein utilize hybrid organic-inorganic halometallates with a dication, histammonium halide (HistNH₃), which has two different cation moieties, one primary ammonium cation and one imidazolium cation. Since the two cations have different steric profiles and vastly different hydrogen-bonding patterns, major differences in cation placement within the crystalline framework can be expected, and, as such, upon changing the B-site cation identity it would be possible to synthesize metallate materials that crystallize in lower symmetry space groups.

The halometallates of the present disclosure can be produced by combining equal parts of a metal halide with histammonium dihydrochloride. In some aspects, the two are combined in an acidic solution.

The compositions of the present disclosure can be provided in a single-crystalline state or as part of a porous film or non-active polymer composite. The halometallate crystals may be deposited by processes such as solution phase deposition, including spin coating, ink jet printing and the like.

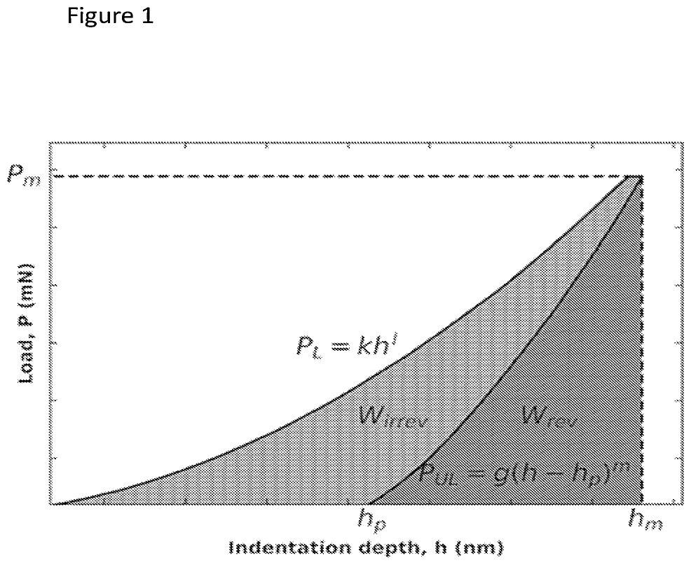

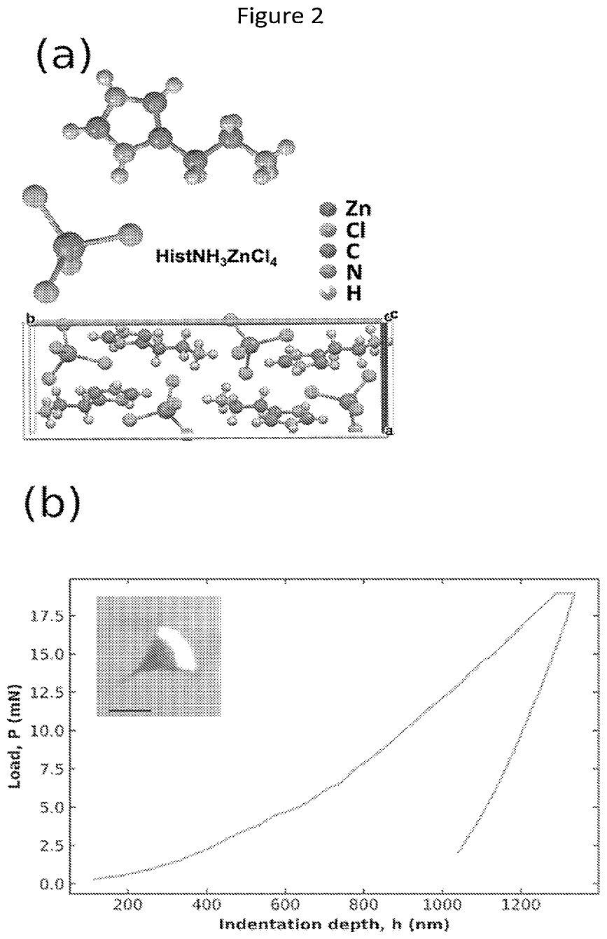

The halometallates may be provided as suspensions within a non-active polymer composite or as a deposit on or within a porous polymer composite film. Suitable polymers include polyvinylidene fluoride (PVDF), polydimethylsiloxane (PDMS), polydiethylsiloxane (PDES), polymethylphenylsiloxane (PMPS), polyvinylidene chloride (PVDC), cellulose acetate, ethylene vinyl acetate, high density polyethylene, polycarbonate, polyester (PET or PEN), polylactic acid, polyurethane, polyvinyl alcohol, polyvinyl butyral, polyvinyl chloride, polyvinyl fluoride, polypropylene, polystyrene, low density polyethylene, cellulose, polytetrafluoroethylene (PTFE), polyimide, melamine, nylon, or combinations thereof. The Examples herein set forth three example organic-inorganic hybrid metalate (OIHM) materials and their piezoelectric characteristics in either the single-crystalline state or in porous composite films (with PVDF). Both the Zn and Co based crystalline material samples exhibited a piezoelectric properties. The HistNH₃ZnCl₄ displayed a high overall piezoelectric response in crystal and in composites. Due to the isolation of metal centers from one another throughout the overall crystalline structure, the materials herein are referred to as OIHMs rather than 0D halide perovskites, which is the more common nomenclature. Four OIHMs (HistNH₃CoCl₂, HistNH₃CuCl₂, HistNH₃ZnCl₂, and HistNH₃NiCl₂) were synthesized herein (FIG. 1), by mixing equimolar quantities of histammonium dichloride and metal (II) chloride in concentrated HCl. Slow evaporation of the solution led to the growth of X-ray quality single crystals (FIG. 1).

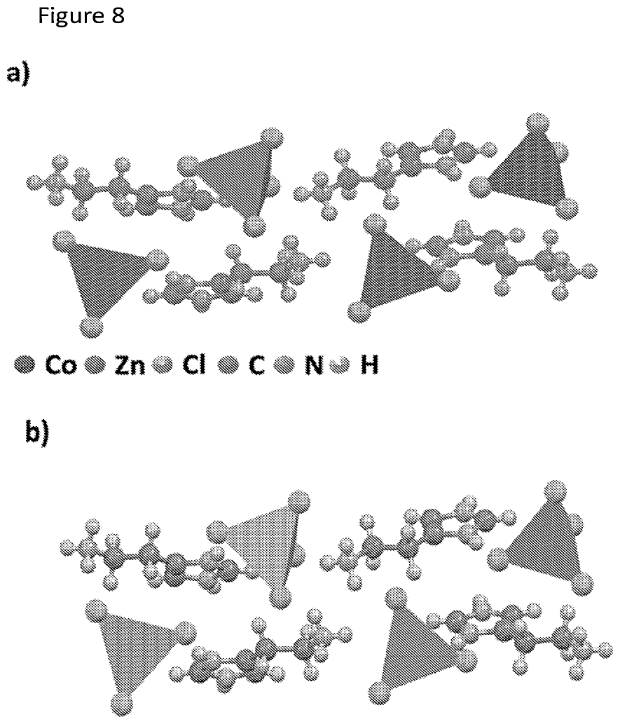

Both HistNH₃ZnCl₄ and HistNH₃CoCl₄ are isostructural (FIG. 8) and were both found to crystallize in a non-centrosymmetric space group (Pna21). They are comprised of individual metal atoms bonded to four (4) chloride ions that yield a tetrahedral dianionic chlorometallate that is charge-balanced by the dicationic distammonium. The imidazolium cations pack to maximize π-π interactions and are spaced ˜3.57 Å, with the ammonium cations packing as far as possible from the other cations. Each MCl₄ (M=Zn, Co) anionic tetrahedron participates in three hydrogen bonds: one of the Cl ligands participates in two hydrogen bonding interactions with two separate histammonium imidazole N—H groups, with an N—Cl—N H-bond angle of ˜156° and a Hydrogen bond distance of ˜3.13 Å, while the Cl ligand nearest the first one participates in a Hydrogen bond interaction with a nearby histammonium amine N—H group with a Hydrogen bond distance of ˜3.21 Å. In each unit cell, there are four MCl₄ tetrahedra, four histammonium molecules, and in HistNH₃ZnCl₄ and HistNH₃CoCl₄ a total of 5 hydrogen bonding interactions per unit cell.

The HistNH₃CuCl₄ material crystallized (FIG. 8) in a centrosymmetric space group (P21/c) and exhibited a unique structure, incorporating one square-planar and one square pyramidal metal center linked through a bridging chloride ligand with a Cu—Cl—Cu bond angle of 163.16°. In the square pyramidal Cu center one longer Cu—Cl bond (˜2.309 Å), two medium Cu—Cl bonds (˜2.31 Å average), one short Cu—Cl bond (˜2.272 Å) are present at the pyramid base, while the apex Cu—Cl bond (2.952 Å) connecting the two metal polyhedra is much longer. In the square planar Cu center two longer Cu—Cl bonds (˜2.304 Å), and two shorter Cu—Cl bonds (˜2.268 Å) are present at the pyramid base and are present, with the shortest Cu—Cl bond (2.967 Å) connecting the two metal. One histammonium cation is present for each of the dianionic chlorometallate tetrahedra, with each cation packing to maximize hydrogen bonding, with a total of 24 hydrogen bonding interactions per unit cell.

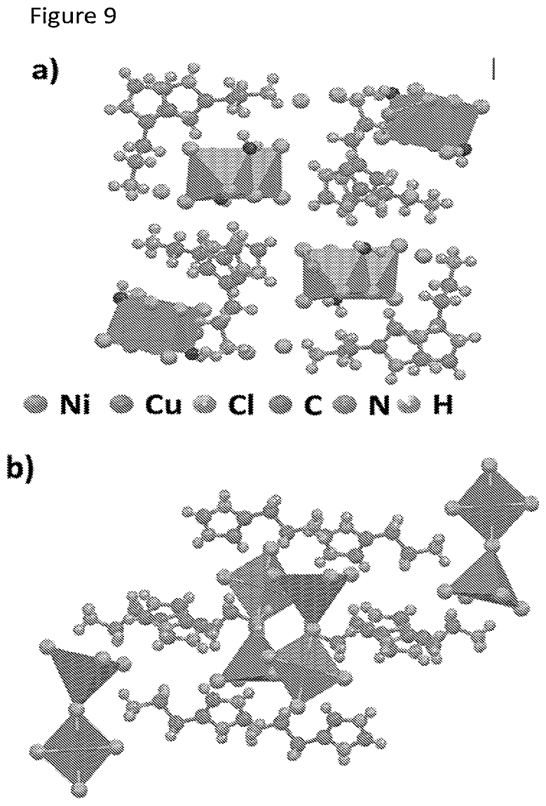

The HistNH₃Ni(H₂O)Cl₄ material crystallized (FIG. 9) in a centrosymmetric space group (P21/c) and exhibited a typical 1-D structure, with metal octahedra extending in long charge-compensated wires. Atypical in this structure, however, is the inclusion of a water molecule coordinated to each Ni center, as is the presence of an uncoordinated Cl ion. The Cl ion is situated near to the coordinated water, as if the water had just recently displaced the water. In the wires, the Ni—Ni bond distance is 3.446 Å, the Ni—Cl bond distances for the shared Cl ligands are 2.394 Å on average, while the unshared Ni—Cl bond has a length of 2.412 Å. The Ni—O bond distance in the wires is 2.131 Å, and the N—Cl distance between the ammonium cation and the Cl ion is 3.681 Å. The ammonium cations are situated towards other ammonium cations and Cl ions and the imidazolium cation is oriented between the NiCl₅(H₂O) wires with π-π stacking between imidazolium subunits, with a total of 27 hydrogen bonding interactions per unit cell.

The thermal changes in the Co, Cu, and Zn containing materials was studied with Differential Scanning calorimetry (DSC) as shown in FIGS. 8 and 9. Features of note include an endothermic trough between 40-80° C., which is believed to be indicative of a crystalline phase transition. The copper metallate shows a possible secondary phase transition at 155° C. before an elevated melting point at 190° C., which is higher than melting temperatures of around 170° C. for Co and Zn metallates.

Piezoforce Microscopy (PFM) was performed on the Co, Cu, and Zn containing single crystal samples grown at RT (room temperature). For each of the materials, it was difficult to consistently see a response displacement from the sample at applied voltages. It is believed this has to do with difficulties in sample mounting, as the crystals were very brittle and difficult to handle, which led to poor electrical contact in some casesDuring the course of measurement, only the Zn-containing metallate was stable to testing and storage in ambient atmosphere, which further made PFM testing on the single-crystalline substrates difficult.

The HistNH₃MCl₄ materials exhibited different piezoelectric responses compared to the PVDF reference, which was annealed at 100° C. and measured according to literature methods. In these trials, the HistNH₃CuCl₄ material was the lowest performing overall (0.28 pm/V), while the HistNH₃ZnCl₄ material exhibited the largest average piezoelectric response, at 22.6 pm/V and HistNH₃CoCl₄ yielded a larger piezoelectric response than PVDF, but much lower than for HistNH₃ZnCl₄.

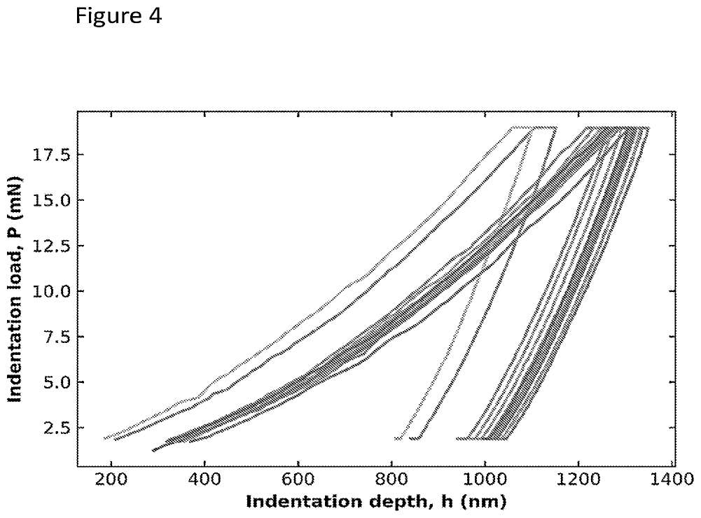

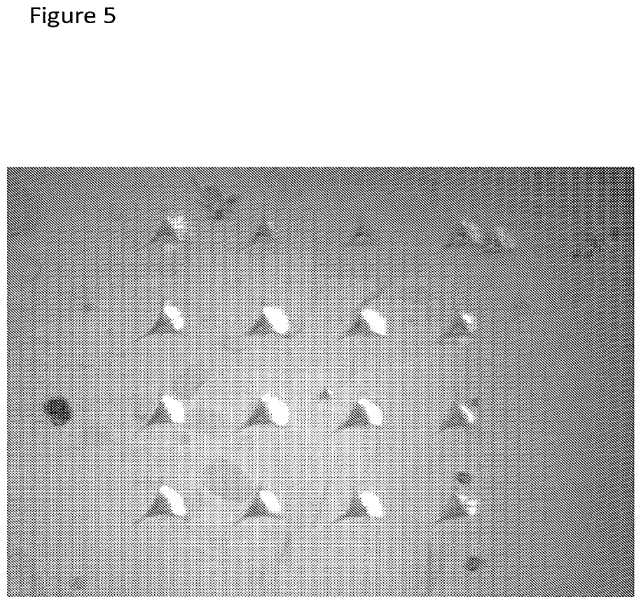

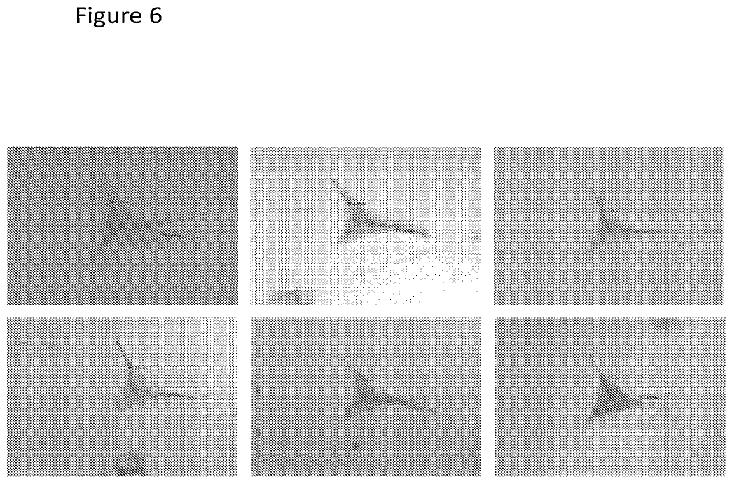

To have a reliable way of testing the piezoelectric responses, HistNH₃MCl₄/PVDF composite materials were made by drop-casting and peeling the films post-annealing. The metallate to PVDF ratio, annealing temperatures, and times were optimized by choosing the compositions and temperatures that led to the easiest-to-peel and handle films after fabrication. The easily-handled films were then tested by PFM. For HistNH₃ZnCl₄. PVDF the composite was 8.6 wt % HistNH₃ZnCl₄ and it was annealed in an oven at 100° C. for 15 minutes, for HistNH₃CuCl₄·PVDF the composite was 5.1 wt % HistNH₃CuCl₄ and it was annealed in an oven at 150° C. for 15 minutes, and for HistNH₃CoCl₄·PVDF the composite was 5.1 wt % HistNH₃CoCl₄ and it was annealed in an oven at 180° C. for 15 minutes. Of the composite materials, the HistNH₃ZnCl₄·PVDF composite films at 8.6 wt % were more resistant to water-induced film degradation than the other composites, which required storage in dry atmosphere after fabrication. Additionally, the HistNH₃CuCl₄·PVDF and HistNH₃CoCl₄·PVDF composite with higher than 5.1 wt % metallate was not easily peeled and so could not be used. The 60×60 topography images shown in the insets of FIG. 4 were taken using contact mode at a scan rate of 0.1 Hz. A linear regression analysis was carried out for each measurement. The slope of the voltage vs displacement was taken as an estimation of the d₃₃ coefficient. FIG. 4 reports the average of the slopes from the 10 different measurements and their standard deviation. To remove the background noise from the measurements, a single measurement was performed on a non-piezoelectric sample (a glass slide) using the same parameters. The measured response of the non-piezoelectric sample was subtracted out of the signals from the polymer composites.

From the PFM measurements on the polymer/metallate composite materials, a similar trend to that observed with single crystalline samples was observed. The HistNH₃ZnCl₄·PVDF composite yielded the highest piezoelectric response (9.6±2.4 pm/V), HistNH₃CoCl₄·PVDF yielded a response marginally better response than the reference PVDF film (5.2±1.3 pm/V). The HistNH₃CuCl₄·PVDF composite yielded no improvement over unmodified PVDF films (4.4±1.2 pm/V). Poling wasn't performed on composite materials, but the amount of matallate material added to the PVDF is at most 8.6% (in the case of HistNH₃ZnCl₄·PVDF), the performance enhancement to PVDF is not negligible.

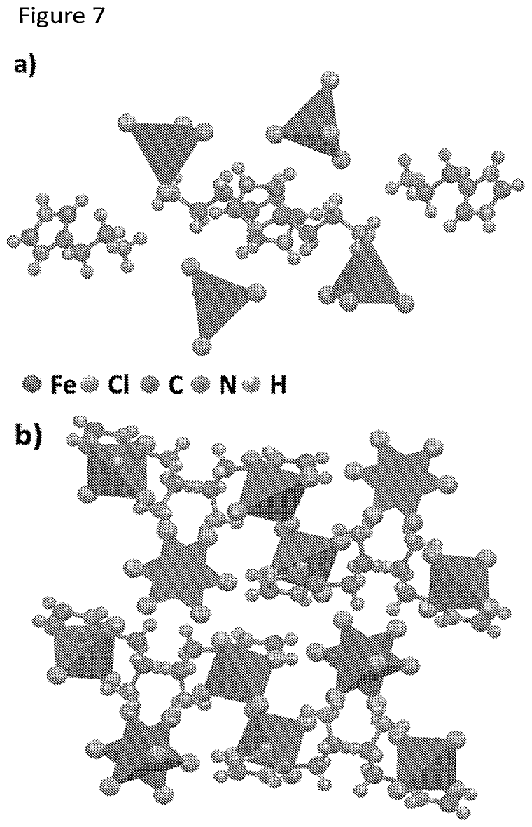

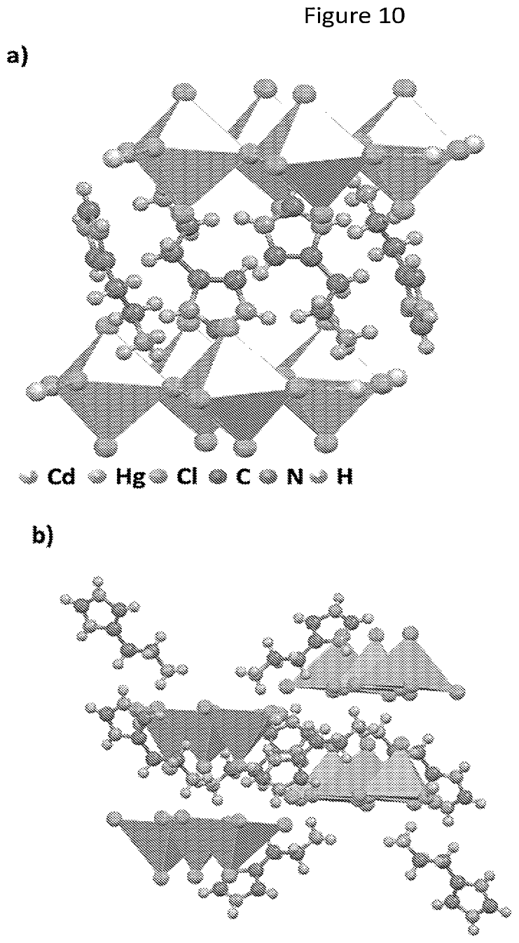

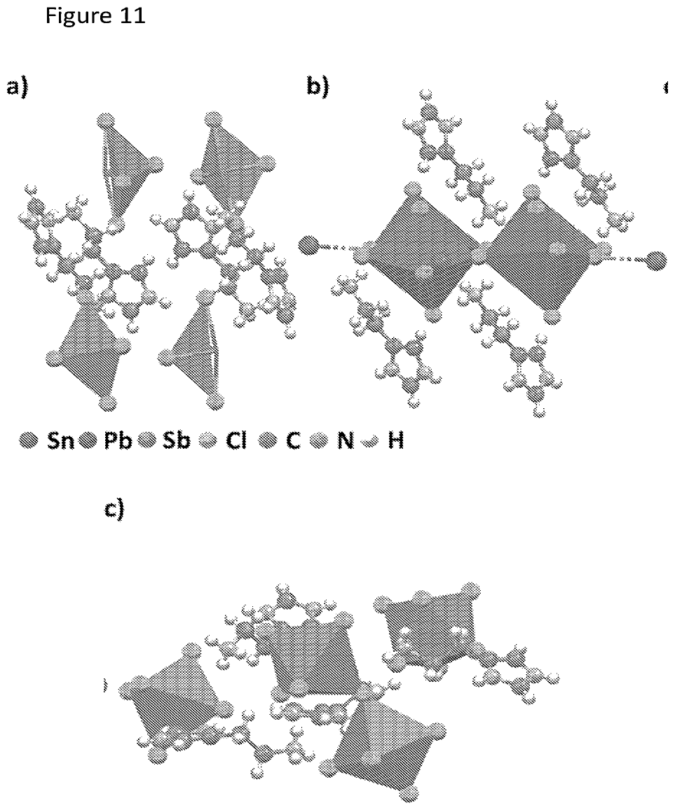

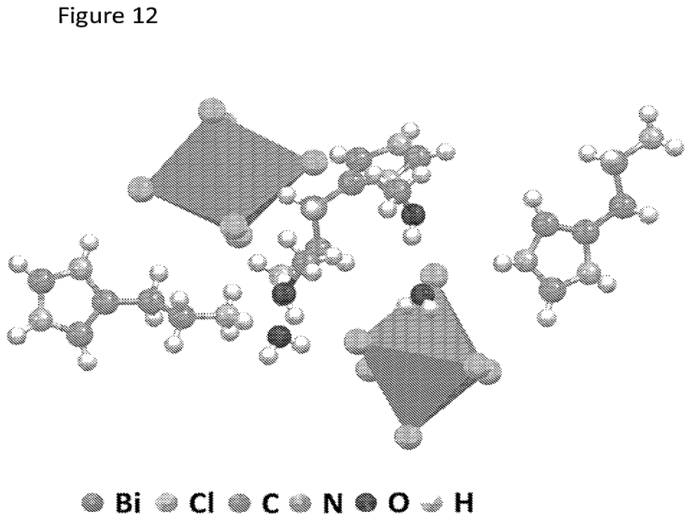

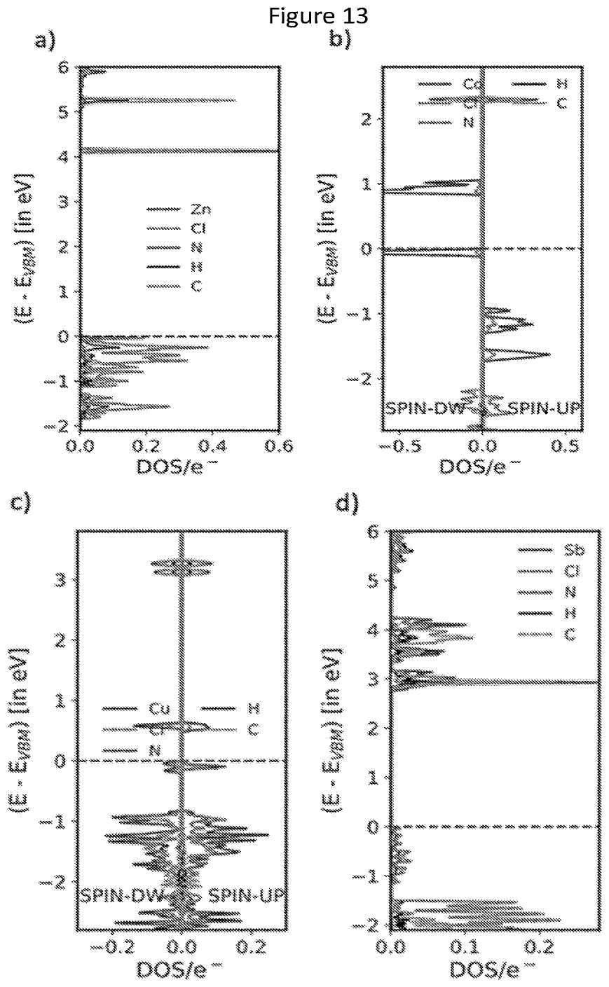

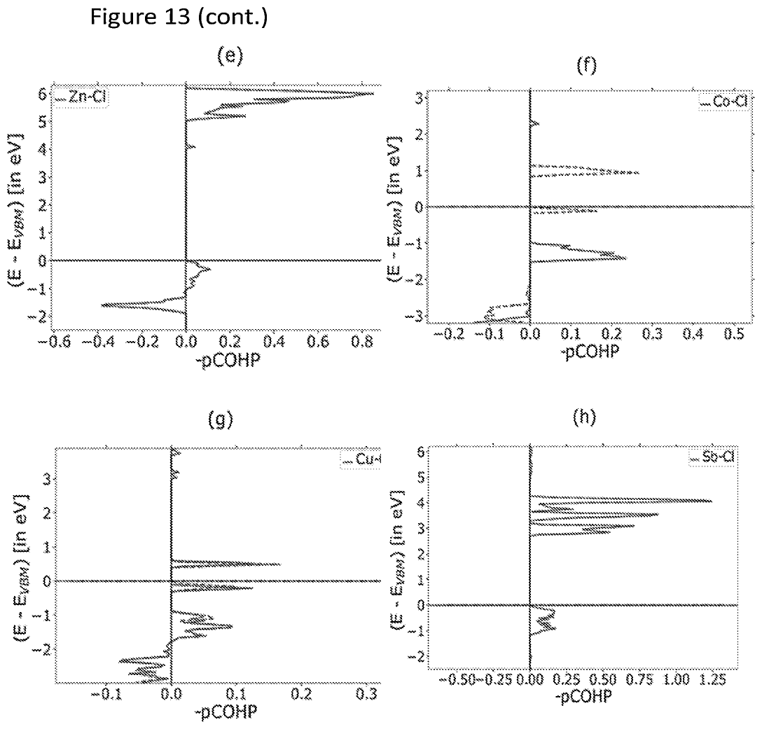

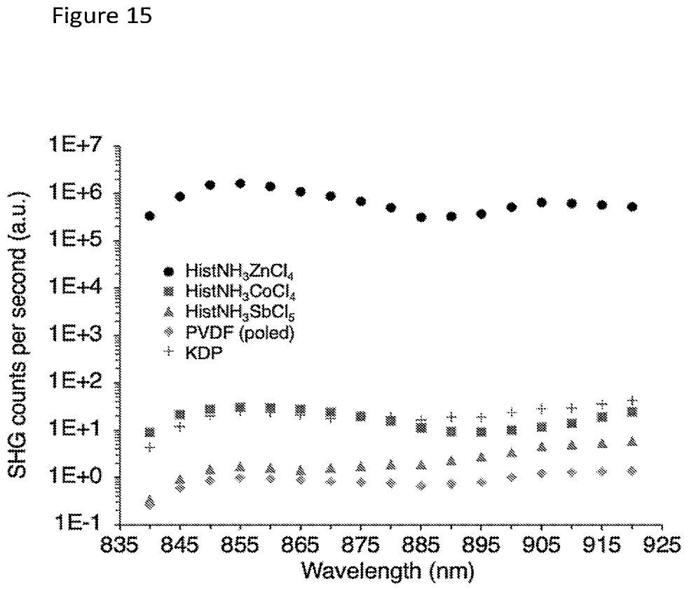

FIGS. 8-12 also demonstrate successful production of halometallates. The HistNH₃FeCl₄ material crystallized from an equimolar mixture of FeCl₂ and histammonium dihydrochloride (FIG. 7a) in a centrosymmetric space group (P2₁/c) and in stacked networks of a typical 0-D structure, with distorted metal-trigonal pyramidal dianions separated by histamine dications. The colorless HistNH₃CdCl₄ material crystallized from an equimolar mixture of CdCl₂ and histammonium dihydrochloride (FIG. 10a) in a centrosymmetric space group (P2₁/c) and exhibited a stacked network of corrugated 2-D structures, with metal octahedra sharing chloride ligands and the colorless HistNH₃HgCl₄ material crystallized from an equimolar mixture of HgCl₂ and histammonium dihydrochloride (FIG. 10b) in a centrosymmetric space group (C2/c) and exhibited stacked networks of a corrugated 1-D structure, with each of the metals bound to five Cl ligands in a distorted square pyramidal geometry. The colorless HistNH₃SnCl₄ material crystallized from an equimolar mixture of SnCl₂ and histammonium dihydrochloride (FIG. 11a) in a centrosymmetric space group (P21/c) and exhibited stacked networks of a 0-D structure, the colorless HistNH₃PbCl₄ material crystallized from an equimolar mixture of PbCl₂ and histammonium dihydrochloride (FIG. 11b) in a centrosymmetric space group (P2₁/c) and exhibited a stacked network of corrugated 2-D structures, with metal octahedra sharing chloride ligands, and the colorless HistNH₃SbCl₅ material crystallized from an equimolar mixture of SbCl₃ and histammonium dihydrochloride (FIG. 15c) in a non-centrosymmetric space group (P2₁2₁2₁) and exhibited a stacked network of corrugated 1-D structures, with metal octahedra sharing two chloride ligands. The colorless (HistNH₃)₃(BiCl₆)₂ material crystallized from an equimolar mixture of BiCl₃ and histammonium dihydrochloride (FIG. 12) in a centrosymmetric space group (P-1) and exhibited stacked networks of a 0-D structure.

### Piezoelectric Devices

Provided herein are also piezoelectric devices that include the halometallate compounds described herein. The piezoelectric devices may also include at least one electrode. It is an aspect of the present disclosure that the compounds described herein exhibit piezoelectric charateristics. Accordingly, affixing or contracting an electrode or electric conductor to the halometallates as set forth herein allows for the transport or collection of charge generated by the halometallate. In some aspects, the halometallate may be presented as a layer in contact with an electrode. The halometallate may include a surface or surface area in contact or connected, at least in part, to an electrode, such as a proximal surface and a distal surface or a top surface and a bottom surface. The piezoelectric devices may include two electrodes in contact with different surfaces of the halometallate. In some aspects, electrodes may sandwich a halometallate, such as being contacted or connected to a top and bottom surface thereof.

The piezoelectric devices of the present disclosure may also include a substrate or an inert surface. The substrate or inert surface may support or hold in place the electrode. The substrate may be of a glass or polymer composition. The substrate may be of Kaptan, polyethylene terephthalate (PET), polyethylene naphthalate (PEN), cellulose, glass, or a similar silica and/or polymer based substance. Optionally, the piezoelectric device may include a halometallate layer in contact or connected to an electrode, with the electrode layered onto the substrate or inert surface. In further aspects, the piezoelectric device may be of four layers: a first and third layer being electrode layers with a second layer therebetween of a halometallate and the third layer being further layered on a substrate or inert surface. It will be appreciated that one or more of the layers may be repeated, such as the halometallate and electrodes. In some aspects a substrate or inert layer may be inserted between to isolate the piezoelectric/electrode responses.

The piezoelectric devices of the present disclosure include halometallates as described herein. In some aspects, the halometallate is a layer or multiple layers within the piezoelectric device. The halometallate layer may be of only one or more halometallate compounds as set forth herein. In some aspects, the halometallate layer may include one or more halometallate compounds in an inert flexible material. In some aspects, the inert flexible material may be an inert polymer or polymer composite. The piezoelectric devices of the present disclosure may further be incorporated as suspensions within a non-active polymer composite or as a deposit on or within a porous polymer composite film. Examples of suitable polymers are provided herein. In some aspects, the halometallate layer is provided as a halometallate composite layer that include one or more halometallates on or within a non-active polymer or polymer composite. For example, as set forth herein, HistNH₃MX halometallates were provided on PVDF films.

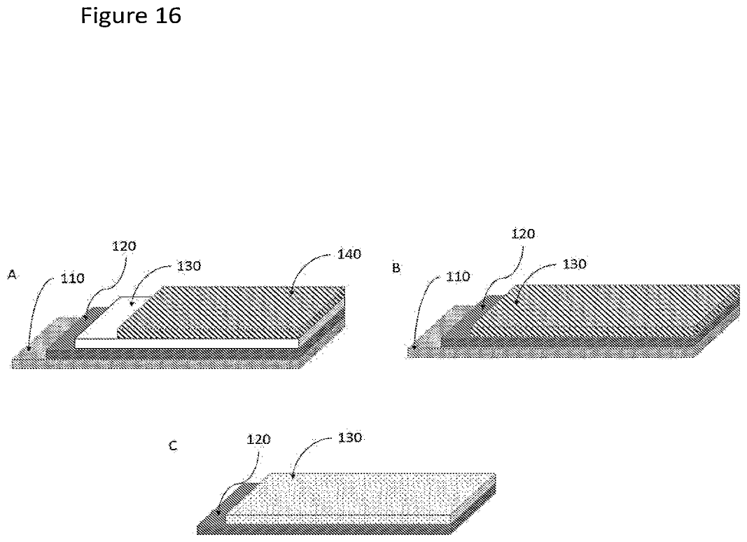

FIG. 16 sets forth three arrangement for piezoelectric devices. In A is depicted a substrate 110 with a first electrode 120 layered thereon. On the opposing side of the electrod 120 is layered a halometallate layer 130, which may include halometallate crystals and/or halometallate composite of halometallate crystals and a non-reactive polymer composite. Atop the halometallate layer 130 is a second electrode 140. B) shows the same arrangement without the second electrode 140 and C) shows the same without the second electrode 140 and the substrate 110.

The piezoelectric devices may be applied or adapted to numerous applications, such as vibrations sensors, energy harvesters, piezoelectric motors, instrument pickups, microphones, actuators, and similar. The piezoelectric devices may similarly be adapted into piezoelectric speakers, amplifiers, accelerometers, and surgical cutting. It will also be apparent that the present disclosure includes methods of making and using both the halometallate compositions and the piezoelectric devices as described herein. A facet of the present disclosure is the identification that the halometallate compositions possess piezoelectric characteristics, and as such, methods applying the compounds to utilize such characteristics are included.

### EXAMPLES

Provided in the present disclosure are novel hybrid organic-inorganic halometallates with a dication, histammonium dichloride (HistNH₃), which has two different cation moieties, one primary ammonium and one imidazolium cation

Reagents used as received from the following chemical suppliers: Hydrochloric Acid, 36-38% from VWR, Hydrobromic and Hydroiodic acids 47% from Beantown Chemical. Histamine Dihydrochloride was purchased from Matrix Scientific and the transition metal chlorides from various sources.

### Materials and Solutions Preparation

The various Histammonium HOIP materials were synthesized by dissolving the transition metal halides MCl (M=Cu, Co, Zn) (1.0 mmol) and 1H-Imidazole-4-ethanamine Dihydrochloride in 1 mL of Hydrochloric acid in a capped 2 mL vial. The contents were warmed to thorough dissolution before being left to crystallize over 12 hours. The single crystals samples prepared for XRD were left to grow from between 48-181 hours to ensure sufficient size.

### Metallate Composite Film Preparation

Composite films used for PFM measurements were prepared by dissolving 10 wt % PVDF and 1.8-8.6 wt % MCl following a weight-percent method involving 1 mL or 1.1 g of DMSOComposites containing PVDF are annealed at 160° C. for 20 minutes.

### Single-Crystal Characterization

Single crystal data of the above compounds were collected on a Bruker D8 Venture κ-axis diffractometer with Mo Kα radiation (0.71073 Å) at 90.0 K. The crystal structures were solved by the direct method and refined using the F2 method and the Shelxtl-XP program. Differential scanning calorimetry measurements were acquired on a TA-Instruments TRIOS DSC 2500 from 0 to 200° C.

### PFM Characterization

Each sample was mounted onto a polished aluminum puck using carbon sticky tape as a conductive adhesive. Piezoresponsive force microscopy (PFM) was performed on all samples as well as a Polyvinylidene difluoride (PVDF) reference sample at 10 different locations using a Bruker Dimension Icon mounted with a SCM-PIT-V2 Platinum-iridium coated conductive tip. To determine the measured displacement of the surface, the tip was calibrated using fused silica to determine the deflection sensitivity. A thermal tuning procedure was also performed to determine the quality factor of the tip (Q =184). A single measurement at a location consisted of measuring the displacement of the sample when applying 0,2,4,6,8, and 10 V by scanning a 500×167 nm∧2 area at an applied load of 68 nN. At each voltage, the response of the sample was taken to be the average response of the resulting image (averaging over ˜22,000 data points).

### Synthesis of Organic Inorganic Hybrid Materials (OIHMs)

Organic Inorganic Hybrid Materials (OIHMs) were synthesized by mixing equimolar quantities of histammonium dichloride and metal chloride in either concentrated HCl, water acidified with HCl, water/methanol mixtures acidified with HCl, or DMSO. Either slow evaporation of the solution or slow cooling of a concentrated solution led to the growth of X-ray quality single crystals.

A pale orange HistNH₃FeCl₄ material crystallized from an equimolar mixture of FeCl₂ and histammonium dihydrochloride (FIG. 7a) in a centrosymmetric space group (P2₁/c) and in stacked networks of a typical 0-D structure, with distorted metal-trigonal pyramidal dianions separated by histamine dications.

Each Fe center is bonded to four Cl ligands, with three shorter Fe—Cl bonds (between 2.2826 and 2.3480 Å), and one longer Fe—Cl bond (2.4091 Å). There were three smaller Cl . . . Fe . . . Cl bond angles (98.44, 99.32, and 101.73°), two angles at 112.73° and 113.35°, and one larger angle of 125.23°. In each unit cell, there are four MCl₄ and four histammonium molecules, a total of 5 hydrogen bonding interactions per histammonium, to yield 20 hydrogen-bonding interactions within and between each unit cell. The bond angles observed here for [FeCl₄]²⁻ are substantially different than those observed in past studies on FeCl₄ OIHM materials, in which each exhibited a tetrahedral geometry.

When FeCl₃ was combined with histamine in an equimolar ratio, we aimed to form FeCl₅ dianions, however, a yellow HistNH₃(FeCl₄)₂ material crystallized (FIG. 7b) in a centrosymmetric space group (Pbca). The HistNH₃(FeCl₄)₂ was comprised of two distorted [FeCl₄]⁻ anion tetrahedra that are charge balanced by the histammonium dication. All eight Fe—Cl bond lengths are similar, and range between 2.1833 Å and 2.2119 Å. The Cl . . . Fe . . . Cl bond angles range from 105.74° at the smallest to 112.67°. Only one hydrogen-bonding interaction is present in each charge balanced structure, between the NH₃ cation and one of the Cl ligands. The FeCl₄ dianions in this structure were typical distorted tetrahedra, in comparison to other Fe^{III}Cl₄ tetrahedra.

Both colorless HistNH₃ZnCl₄ and deep blue HistNH₃CoCl₄ materials are isostructural (FIG. 8a, 8b) and were both found to crystallize from an equimolar mixture of MCl₂ (M=Co, Zn) and histammonium dihydrochloride in a non-centrosymmetric space group (Pna2₁), in a stacked network of a 0-D structure.

They are comprised of individual metal atoms bonded to four (4) chloride ions that yield a tetrahedral dianionic chlorometallate that is charge-balanced by the dicationic histammonium. The M—Cl bond distances range from 2.2540 Å to 2.2891 Å when M=Zn and from 2.2589 Å to 2.2883 Å when M=Co. There are two Cl—M—Cl bond angles that are below the ideal tetrahedron angles (106.64° and 107.24° when M=Zn and 106.20° and 107.94° when M=Co) and two that are above (111.15° and 114.16° when M=Zn and 111.95° and 114.67° when M=Co). The imidazolium cations pack to maximize π-π interactions and are spaced ˜3.57 Å, with the ammonium cations packing as far as possible from the other cations. Each MCl₄ (M=Zn, Co) anionic tetrahedron participates in three hydrogen bonds: one of the Cl ligands participates in two hydrogen bonding interactions with two separate histammonium imidazole N—H groups, with an N . . . Cl . . . N H-bond angle of ˜156° and a hydrogen bond distance of ˜3.13 Å, while the Cl ligand nearest the first one participates in a hydrogen bond interaction with a nearby histammonium amine N—H group with a hydrogen bond distance of ˜3.21 Å. In each unit cell, there are four MCl₄ tetrahedra, four histammonium molecules, and in HistNH₃ZnCl₄ and HistNH₃CoCl₄ a total of 3 hydrogen bonding interactions per histammonium, for a total of 12 hydrogen-bonding interactions within and between each unit cell. The structural parameters of both materials were similar to other Zn^{II}Cl₄ and Co^{II}Cl₄ tetrahedra reported in the literature.

The yellow HistNH₃Ni(H₂O)Cl₄ material crystallized from an equimolar mixture of NiCl₂ and histammonium dihydrochloride (FIG. 9a) in a centrosymmetric space group (P2₁/c), however the crystals were 4-fold twinned by pseudo-merohedry.

They exhibited stacked networks of a typical 1-D structure, with metal octahedra extending in long charge-compensated wires. Atypical in this structure, however, is the inclusion of a water molecule coordinated to each Ni center, as is the presence of an uncoordinated Cl ion. The Cl ion is situated near to the coordinated water. In the wires, the Ni . . . Ni bond distances alternate between 3.446 Å and 3.456 Å, the Ni—Cl bond distances for the shared Cl ligands are 2.394 Å on average, while the unshared Ni—Cl bond has a length of 2.412 Å. The Ni—O bond distance in the wires is 2.132 Å, and the N—Cl distance between the ammonium cation and the Cl ion is 3.681 Å. The ammonium cations are situated towards other ammonium cations and Cl ions and the imidazolium cation is oriented between the NiCl₅(H₂O) wires with π-π stacking between imidazolium subunits, with a total of 27 hydrogen-bonding interactions within and between each unit cell. Others have observed other types of linear 1-D chloronickelates, chloronickelates with Cl and OH₂ ligands, but the most commonly observed chloronickelate geometry is tetrahedral.

The green HistNH₃CuCl₄ material crystallized from an equimolar mixture of CuCl₂ and histammonium dihydrochloride (FIG. 9b) in a centrosymmetric space group (P2₁/c), but were non-merohedric twins. They exhibited a unique structure, incorporating one square-planar and one square-pyramidal metal center linked through a bridging chloride ligand with a Cu . . . Cl . . . Cu bond angle of 163.16°. In the square pyramidal Cu center one longer Cu—Cl bond (2.3093 Å), two medium Cu—Cl bonds (˜2.31 Å average), one short Cu—Cl bond (2.2716 Å) are present at the pyramid base, while the apex Cu—Cl bond (2.952 Å) connecting the two metal polyhedra is much longer. In the square planar Cu center two longer Cu—Cl bonds (˜2.304 Å average), and two shorter Cu—Cl bonds (˜2.268 Å average) are present. One histammonium cation is present for each of the dianionic chlorometallate tetrahedra, with each cation packing to maximize hydrogen bonding, with a total of 24 hydrogen-bonding interactions within and between each unit cell. The mixed square-pyramidal/square-planar geometry observed in the structure here is not typical of the structures previously observed for CuCl₄ based OIHMs, as others have observed a variety of structures dependent mostly on the organic cation identity. In one account, a relatively similar structure was found when the dication (CH₂)₃(NH₃)₂ was used with CuCl₄, which resulted in an additional Cu—Cl bond and a zig-zag square-pyramidal bonding pattern.

The colorless HistNH₃CdCl₄ material crystallized from an equimolar mixture of CdCl₂ and histammonium dihydrochloride (FIG. 10a) in a centrosymmetric space group (P2₁/c) and exhibited a stacked network of corrugated 2-D structures, with metal octahedra sharing chloride ligands.

Each octahedron is slightly tilted in reference to its neighbors, and in the opposite orientation. Each histammonium dication is oriented head-to-tail with its organic spacer neighbor. In the 2-D sheets, each of the unshared Cl ligands in the plane of the 2-D sheet have a Cd—Cl bond distance of 2.5424 Å, the shared Cl ligands have bond distances of 2.6576 Å and 2.6410 Å, the Cl ligands orthogonal to the 2-D sheet have bond distances of 2.7576 Å and 2.7199 Å. The Cd . . . Cl . . . Cd bond angle of one of the shared Cl is 163.11°, the Cl . . . Cd . . . Cl angle is 174.53°, while the Cd . . . Cl . . . Cd bond angle of the other shared Cl is 179.27° and the Cl . . . Cd . . . Cl bond angle is 171.83°. Hydrogen-bonding interactions are present with each of the protonated nitrogens, one each for the imidazolium cations and four for the primary ammonium cations. Since there are four histammonium spacers per unit cell and six hydrogen bond interactions per histammonium, there are 24 hydrogen-bonding interactions within and between each unit cell. The 2-D structure found here is the typical structure found in chlorocadmiates.

The colorless HistNH₃HgCl₄ material crystallized from an equimolar mixture of HgCl₂ and histammonium dihydrochloride (FIG. 10b) in a centrosymmetric space group (C2/c) and exhibited stacked networks of a corrugated 1-D structure, with each of the metals bound to five Cl ligands in a distorted square pyramidal geometry. Two of the Cl basal ligands are shared, two basal Cl ligands unshared, and the apical Cl ligand unshared as well. The apical Hg—Cl bond distance is 2.632 Å, the shared basal Hg—Cl bond distance is 2.4184 Å, the shared Hg . . . Cl . . . Hg bond angle is 148.72° and the Cl . . . Hg . . . Cl bond angle is 158.57°, and the unshared Hg—Cl bond distance is 2.857 Å with a Cl . . . Hg . . . Cl bond angle of 164.34°. Hydrogen-bonding interactions are present with each of the protonated nitrogens, with each imidazolium cation participating in one interaction and four interactions present for the primary ammonium cations. Since there are eight histammonium spacers per unit cell and six hydrogen-bond interactions per histammonium, there are 48 hydrogen-bonding interactions within and between each unit cell. Others have reported tetrahedral chloromercurate structures, but others have observed a similar corrugated 1-D structure from HgCl₄.

The colorless HistNH₃SnCl₄ material crystallized from an equimolar mixture of SnCl₂ and histammonium dihydrochloride (FIG. 11a) in a centrosymmetric space group (P21/c) and exhibited stacked networks of a 0-D structure.

Each Sn center is bonded to four Cl ligands in a distorted seesaw geometry, with two shorter Sn—Cl bonds (between 2.5354 and 2.5625 Å), and two longer Sn—Cl bonds (2.7271 and 2.8406 Å). The Clₐₓ . . . Sn . . . Clₐₓ bond angle was 167.31°, while each of the other Cl . . . Sn . . . Cl bond angles was between 84.17 and 87.93°. Three of the Cl ligands participates in hydrogen bonding with the histammonium dication. In each unit cell there are four MCl₄ and four histammonium molecules with a total of five hydrogen bonding interactions per histammonium (three with the ammonium and two with the imidazolium cation), to yield 20 hydrogen-bonding interactions within and between each unit cell. The bond angles observed here for SnCl₄²⁻ are substantially different than those observed in previous studies on 0-D Sn OIHM materials, in which those with Br and I each exhibited an octahedral geometry. In SnClₓ OIHMs, many others have reported exclusive formation of [SnCl₆]²⁻ octahedra after using SnCl₂, which results from Sn²⁺ to Sn⁺⁴ oxidation, or after using SnCl₄. In a Cambridge Structural Database search, only one other SnCl4⁻² with a seesaw geometry was found.

The colorless HistNH₃PbCl₄ material crystallized from an equimolar mixture of PbCl₂ and histammonium dihydrochloride (FIG. 11b) in a centrosymmetric space group (P2₁/c) and exhibited a stacked network of corrugated 2-D structures, with metal octahedra sharing chloride ligands. Each octahedron is tilted in reference to its neighbors, as in the HistNH₃CdCl₄ structure, however, each Pb octahedron is tilted much more, with a Pb—Cl—Pb angle of 150.23° vs. the Pb—Cl—Pb angle of 163.11°. Each histammonium dication is oriented head-to-tail with its organic spacer neighbor, with the ammonium cation in close contact with three Cl ligands. In the 2-D sheets, each of the unshared Cl ligands have Pb—Cl bond distances of 2.7901 and 2.8982 Å, the shared Cl ligands have bond distances of 2.7200 Å and 2.9656 Å, 2.759 Å and 3.0610 Å. The Pb . . . Cl . . . Pb bond angle of the one of the shared Cl is 150.23°, the Cl . . . Pb . . . Cl angle is 170.78°. Hydrogen-bonding interactions are present with each of the protonated nitrogens, one each for the imidazolium nitrogens and three for the primary ammonium cations. Since there are four histammonium spacers per unit cell and five hydrogen bond interactions per histammonium, there are 20 hydrogen-bonding interactions within and between each unit cell. The 2-D structure observed for HistNH₃PbCl₄ was the common n=1 structure, where n is the inorganic layer number, structure reported by others.

The colorless HistNH₃SbCl₅ material crystallized from an equimolar mixture of SbCl₃ and histammonium dihydrochloride (FIG. 11c) in a non-centrosymmetric space group (P2₁2₁2₁) and exhibited a stacked network of corrugated 1-D structures, with metal octahedra sharing two chloride ligands. Each octahedron is therefore tilted in reference to its neighbors, each Sb octahedron is tilted away from its neighbor, with a Sb—C₁—Sb angle of 147.98°. Each histammonium dication is oriented head-to-tail with its organic spacer neighbor, with the ammonium cation in close contact with three Cl ligands and the imidazolium in close contact with two Cl ligands. In each octahedron, three Cl ligands have Sb—Cl bond distances between 2.472 and 2.509 Å and one Cl ligand has a bond distance of 2.822 Å. The Sb . . . Cl bond distance of one of the shared Cl is 2.868 Å., the other is 2.861 Å. Hydrogen-bonding interactions are present with each of the protonated nitrogens, one each for the imidazolium nitrogens and three for the primary ammonium cations. Since there are four histammonium spacers per unit cell and five hydrogen bond interactions per histammonium, there are 20 hydrogen-bonding interactions within and between each unit cell. The corrugated 1-D structure observed for HistNH₃SbCl₅ is similar to a recently reported pentachlorantimonate, and to a histammnoium iodoantimonate.

The colorless (HistNH₃)₃(BiCl₆)₂ material crystallized from an equimolar mixture of BiCl₃ and histammonium dihydrochloride (FIG. 12) in a centrosymmetric space group (P-1) and exhibited stacked networks of a 0-D structure. Also included in the structure are two water and two methanol solvent molecules.

In one of the chlorobismuthates, the Bi center is bonded to six Cl ligands in a distorted octahedron, with two shorter Bi—Cl bonds (2.6512 and 2.6641 Å), two intermediate-length Bi—Cl bonds (2.6925 and 2.7019 Å), and two longer Bi—Cl bonds (2.7556 and 2.7638 Å for example). The Clₐₓ . . . Bi . . . Clₐₓ bond angle was 174.72°, while each of the other Cl . . . Bi . . . Cl bond angles was between 85.68 and 95.74°. In the other chlorobismuthate, the Bi center is also bonded to six Cl ligands in a distorted octahedron, with two shorter Bi—Cl bonds (2.6544 and 2.6824 Å), two intermediate-length Bi—Cl bonds (2.6916 and 2.7032 Å), and two longer Bi—Cl bonds (2.7134 and 2.7781 Å for example). The Clₐₓ . . . Bi . . . Clₐₓ bond angle was 176.133°, while each of the other Cl . . . Bi . . . Cl bond angles was between 84.28 and 95.20°. Each Cl ligand of the two BiCl₆ trianions participates in hydrogen bonding, but the types of hydrogen bonds are different between them. In one BiCl₆, two chlorine atoms hydrogen bond with nearby water molecules, one chloring atom hydrogen bonds with a nearby methanol molecule, and the other three hydrogen bond with histammine molecules. The water molecules also hydrogen bond to histammine molecules or methanol. In the other BiCl₆ group, five of the chlorine atoms hydrogen bond with histammine, but one chlorine is not participating in any hydrogen bonding. In each unit cell there are two MCl₆ and three histammonium molecules. One of the histammines has a total of four hydrogen bonding interactions, one has six, and the other has five. One of the water molecules has three hydrogen bonds and the other has four. One of the methanol molecules has two hydrogen bonds, and the other has three for a total of 27 hydrogen-bonding interactions within and between each unit cell. The bond angles observed here for BiCl₆²⁻ differ from those observed in some previous studies on 0-D Bi OIHM materials, in which the longest Bi—Cl bond lengths here are either longer by <0.05 Å or shorter by >0.13Å than the longest Bi—Cl bond in a BiCl₆³⁻ octahedron. Overall, the crystalline structure of the chlorometallate structures described here changed markedly with a change in metal cation identity (Table 1).

TABLE 1
Properties of hybrid inorganic-organic chlorometallates studied here.

| | Metal | | | | Chlorides |
|---|---|---|---|---|---|
| | Cation | Space | Metal Ionic | Material | shared per |
| Material | Charge | Group | Radius (pm)⁵⁵ | Dimensionality | metal atom |
| HistNH₃FeCl₄ | +2 | P2₁/c | 92 | 0-D | 0 |
| HistNH₃(FeCl₄)₂ | +3 | Pbca | 78.5 | 0-D | 0 |
| HistNH₃CoCl₄ | +2 | Pna2₁ | 88.5 | 0-D | 0 |
| HistNH₃ZnCl₄ | +2 | Pna2₁ | 88 | 0-D | 0 |
| [HistNH₃Ni(H₂O)Cl₃]Cl | +2 | P2₁/c | 83 | 1-D | 2 |
| HistNH₃CuCl₄ | +2 | P2₁/c | 87 | 0-D dimers | 1 |
| HistNH₃CdCl₄ | +2 | P2₁/c | 109 | 2-D | 4 |
| HistNH₃HgCl₄ | +2 | C2/c | 116 | Corrugated 1-D | 1 |
| HistNH₃SnCl₄ | +2 | P2₁/c | 102⁵⁶ | 0-D | 0 |
| HistNH₃PbCl₄ | +2 | P2₁/c | 133 | 2-D | 4 |
| HistNH₃SbCl5 | +3 | P2₁2₁2₁ | 90 | Corrugated 1-D | 1 |
| (HistNH₃)₃(BiCl₆)₂ | +3 | P-1 | 117 | 0-D | 0 |

Most of the materials with a divalent metal cation crystallized into the P2₁/c space group, with the non-centrosymmetric Co-(Pna2₁), Zn-(Pna2₁), and centrosymmetric Hg-based ((C2/c)) materials being the only exceptions. None of the materials exhibited a 3-D dimensionality, where all the chloride ligands are shared with neighboring metal centers. Only two materials (based on Cd and Pb) exhibited a 2-D dimensionality, with four chlorides being shared, and two others (Ni and Hg) exhibited a 1-D dimensionality, with two shared chloride ligands. The histammonium dication is much too large to allow 3-D functionality as determined by the Goldschmidt tolerance factor, and using it resulted in mostly 0-D materials, in metals with varying ionic radii. It is likely that the crystal growth conditions played some role in crystal structure formation (especially for the Ni- and Bi-compounds), which makes precise accounting for the effect of the histammonium cation on dimensionality and space-group more difficult.

While the structures of the materials from Table 1 depended on the identity of the metal cation, only three of the solved structures were non-centrosymmetric (HistNH₃CoCl₄, HistNH₃ZnCl₄, and HistNH₃SbCl₄). Since there was interest in the potential piezoelectric properties of these chlorometallates, it was decided decided to study further the piezoelectric properties of the non-centrosymmetric materials. The Zn-, Co-, and Sb-based materials were studied, as well as the centrosymmetric HistNH₃CuCl₄, which was chosen at random to serve as a negative control. PFM was performed on the X-ray quality Co, Cu, Sb, and Zn containing single-crystal OIHM samples grown and maintained at room temperature. For each of the materials, it was difficult to consistently see a response displacement from the sample at the applied voltages. It was decided to attribute this to difficulties we had in sample mounting, as the crystals were highly brittle and damaged easily, which led to poor electrical contact in some cases. Only the Zn containing material grew large enough crystals to measure on a specific face (a-c face). Table 2 shows results of successful PFM measurements from these single-crystal samples. During measurement, only the Zn-containing metallate was stable to testing and storage in ambient atmosphere, which further made PFM testing on the single-crystalline substrates difficult. Nevertheless, when measurements were successfully taken, multiple repetitive sampling events were taken from several sites on the mounted crystal.

TABLE 2
Local piezoelectric response from piezoresponse force microscopy
measurements on unpoled single-crystalline samples of
HistNH₃Mⁿ⁺Clₙ₊₂ (M = Co, Cu, Zn, Sb) compared
to a ferroelectric polymer PVDF reference.

| Material | Local Piezoelectric Response (pm/V) |
|---|---|
| HistNH₃CoCl₄ | 5.17 |
| HistNH₃CuCl₄ | 0.28 |
| HistNH₃ZnCl₄ | 22.6 |
| HistNH₃SbCl₅ | 2.9 |
| PVDF | 2.5 |

To rationalize these results, we performed density functional theory (DFT) calculations using the Perdew-Burke-Ernzerhof (PBE) functional (see Computational details section for more details) for the set of compounds listed in Table 2: HistNH₃CoCl₄, HistNH₃SbCl₅, HistNH₃CuCl₄, and HistNH₃ZnCl₄.

To compare with the experiment by removing any dependency on the structure, the bandgap, charge transfer, net and cation-Cl dipole moments, and d33 constants provided in Table 3 were all calculated with PBE at the experimental geometry. Charge transfer and dipole moments were calculated using DDEC6.

To examine the variation of the key properties with the different geometries, a comparison of the bandgaps and piezoelectric strain tensors were all calculated with the PBE functional but at four different geometries. The four geometries used in this analysis were: (i) experimental; (ii) PBE-partially optimized geometry whereby the volume is fixed but the lattice sites are relaxed; (iii) PBE fully optimized; and (iv) PBE+D3 fully optimized. The comparison between the PBE and PBE+D3 fully optimized geometries were motivated by the fact that the calculated lattice constants and unit-cell volume of the PBE-fully optimized geometries are ca. 10% larger than experiment, which is consistent with the previous literature. On the other hand, the lattice constants and unit-cell volume with PBE+D3-optimized geometries are calculated to be within 2% of experiment. Small variations in the band gap energies and piezoelectric strain tensors were found depending on the geometry; however, the overall trend is the same.

The PBE-calculated bandgaps at the experimental geometry are in good agreement with the measured values, which were determined using UV-Vis-NIR measurements of dropcasted films (Table 3). The observed low energy absorbances in the films of the Co- and Cu-based materials were ascribed to spin-forbidden d-d transitions. The piezoelectric strain constants (dᵢⱼ) were determined from the PBE-calculated elastic (Cᵢⱼ) and piezoelectric stress (eᵢⱼ) tensors using the relation dᵢⱼ=eᵢⱼ×Sᵢⱼ; here, Sᵢⱼ is the inverse of the matrix Cᵢⱼ. As shown in Table 3, the calculated d₃₃ coefficients for HistNH₃ZnCl₄ and HistNH₃CoCl₄ are 10.8 pm/V and 7.4 pm/V, respectively, and 0.0 for HistNH₃SbCl₄ and HistNH₃CuCl₄ (see Tables S11-S22 for computed tensor components). For HistNH₃CuCl₄, the calculated piezoelectric strain coefficients are zero, which is expected because of its centrosymmetric space group (P2₁/c). For HistNH₃SbCl₅, although the d₃₃ value is zero, some of the calculated coefficients are non-zero. This compares well with the experimental findings that the piezoelectric response is weaker than that for HistNH₃ZnCl₄ or HistNH₃CoCl₄ (Table 2), but much stronger than that for the Cu-containing material because PFM often captures some contributions from non-d₃₃ strain tensors (like d₃₁ and d₃₄).

The increased experimentally measured piezoelectric responses in going from HistNH₃SbCl₅ to HistNH₃CoCl₄ and HistNH₃ZnCl₄ is attributed to an increase in the cation-Cl bond dipole moment in these compounds (see Table 3), which ranges from 0.0 D for HistNH₃SbCl₅ to 0.414 D for HistNH₃CoCl₄ and 0.479 D for HistNH₃ZnCl₄. The overall calculated net dipole moment per unit cell is small because of an apparent cancellation in the contributions from HistNH₃ and cation-Cl bond. In general, from this analysis, we identify the larger cation-Cl bond dipole to be the source of the larger measured piezoelectric response in HistNH₃ZnCl₄ compared with HistNH₃CoCl₄.

TABLE 3
Measured bandgap and calculated bandgap, charge transfer, net dipole moment per unit cell, dipole
moment contributed from cation-Cl bond and d₃₃ constant in the Co, Sb, Cu, and Zn metalates.

| | | | | Net Dipole | | | Average |
|---|---|---|---|---|---|---|---|
| | | | Charge | moment | Cation-Cl | Calculated | value of |
| | | | transfer | per unit | bond Dipole | d₃₃ | PFM |
| | Measured | Calculated | (e⁻/metal | cell | moment | constant | response |
| System | bandgap | bandgap | atom) | (Debye) | (Debye) | (pm/V) | (pm/V) |
| HistNH₃CoCl₄ | 0.88 | 0.88 | 0.630 | 0.037 | 0.41 | 7.39 | 5.17 |
| HistNH₃SbCl₅ | 3.22 | 2.88 | 0.957 | 0.0 | 0.000 | 0.00 | 2.9 |
| HistNH₃CuCl₄ | 0.55 | 0.53 | 0.717 | 0.0 | 0.000 | 0.00 | 0.28 |
| HistNH₃ZnCl₄ | 4.13 | 4.15 | 0.635 | 0.048 | 0.48 | 10.76 | 22.6 |

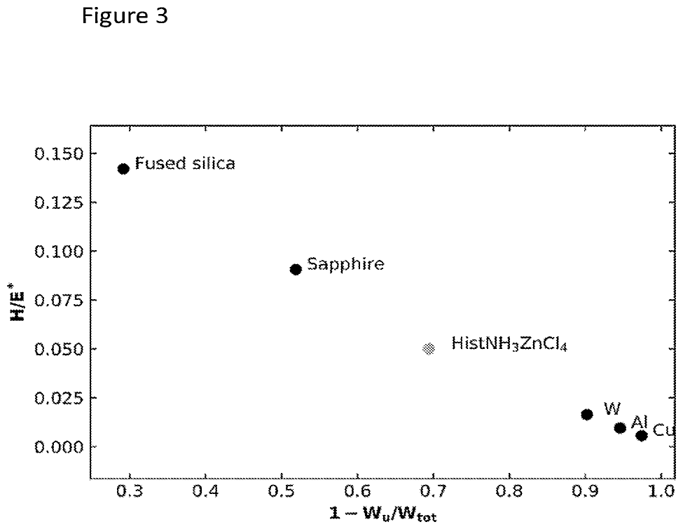

To rationalize the larger dipole moment for HistNH₃ZnCl₄ compared with HistNH₃CoCl₄ and HistNH₃SbCl₅, both the elementwise contribution (partial) of the density of states (pDOS) and the projected crystal orbital Hamilton population (pCOHP) were calculated (see FIG. 3). The pDOS and pCOHP are used to calculate the contributions of each element to the conduction band minimum (CBM) and valence band maximum (VBM). The COHP analysis was performed using LOBSTER.

Both the pDOS and pCOHP indicates that the higher calculated dipole for HistNH₃ZnCl₄, is due to the lower hybridization of the Zn—Cl bond compared with Co—Cl. For HistNH₃ZnCl₄, the VBM is dominated by Cl with a slight contribution from Zn and the CBM is dominated by the N atom of HistNH₃. Similarly, the COHP analysis indicates that the Zn—Cl bonding dominates the valance band edge but is largely absent from the conduction band edge. This is consistent with experimental data in the literature suggesting that in ZnCl₄²⁻, the electron density of the highest occupied molecular orbital lies mainly in a molecular orbital with primarily Cl ligand p-orbital character.

In the isostructural compound HistNH₃CoCl₄, the CBM and VBM are both dominated by Co and Cl. This is consistent with the literature that suggests in CoCl₄²⁻, the highest occupied molecular orbital lies mainly in Co-centered 3d-t₂ orbitals, with little delocalization into the Co—Cl σ bond. In HistNH₃SbCl₅ the VBM and CBM is both dominated by Sb and Cl. This agrees with the literature that the valance band is dominated by the strong hybridization between the occupied Sb-5s and Cl-3p while conduction band is dominated by Sb-5p and Cl-3p. Both the VBM and CBM in the case of HistNH₃CuCl₄ is dominated by Cu and Cl as suggested by pDOS and COHP analysis.

In the results shown in Table 2, the HistNH₃MCl₄ materials exhibited different local piezoelectric responses compared to the PVDF ferroelectric polymer reference, which was annealed at 100° C. in an oven and measured according to literature methods. In these trials, the HistNH₃CuCl₄ material was the lowest performing overall (0.28 pm/V) and very close to zero, as expected, due to the inversion symmetry present in the crystal. This number is not precisely 0, however, because centrosymmetric materials can give very small local piezoelectric response values. The HistNH₃ZnCl₄ material exhibited the largest average local piezoelectric response, at 22.6 pm/V and HistNH₃CoCl₄ yielded a larger local piezoelectric response than PVDF, but much lower than for HistNH₃ZnCl₄. HistNH₃SbCl₅ exhibited a PFM response that was a little higher than the PVDF reference. The difference in performance between the isostructural Zn and Co based materials is discussed in more detail below. Even though these values are notable, the samples here were tested without any poling treatments, due to difficulty in making electrical connection to the single crystals, thus they are likely underestimated. State-of-the-art approaches towards piezoelectric metallates have yielded materials with d₃₃ piezoelectric coefficients of 73 and 76 pm/V for Pb based metallates, 110 pm/V for an Fe based metallate, and 139 and 1540 pm/V for Cd based metallates. While others have suggested that a local inverse piezoelectric response observed from PFM could be extrapolated into a bulk d₃₃ value, which would place HistNH₃ZnCl₄ firmly within the state-of-the-art, we were not able to pole these samples as of yet. Efforts are underway to grow sufficiently large crystals to allow poling and bulk d₃₃ piezoelectric measurement.

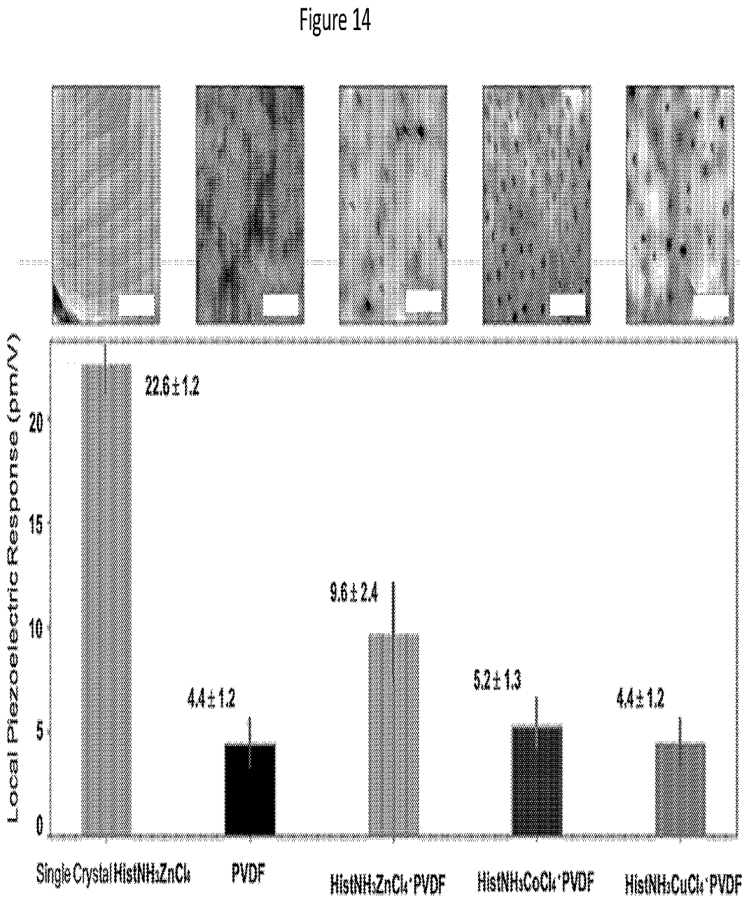

To further investigate whether the piezoelectric response measurements are related to orientation of the mounted single crystals, HistNH₃MCl₄ (M=Cu, Co, Zn)·PVDF composite materials were made by drop-casting, annealing in an oven, and peeling composite films post-anneal to yield a free standing and flexible film. The metallate to PVDF ratio, annealing temperatures, and times were optimized by choosing the compositions and temperatures that led to the easiest-to-peel and handle films after fabrication. The easily handled and non-poled films were then tested by PFM.⁶ For HistNH₃ZnCl₄·PVDF the composite was 8.6 wt. % HistNH₃ZnCl₄ which was annealed at 100° C. for 15 minutes, for HistNH₃CuCl₄·PVDF the composite was 5.1 wt. % HistNH₃CuCl₄ which was annealed at 150° C. for 15 minutes, and for HistNH₃CoCl₄·PVDF the composite was 5.1 wt. % HistNH₃CoCl₄ which was annealed at 180° C. for 15 minutes. Of the composite materials, the HistNH₃ZnCl₄·PVDF composite films at 8.6 wt. % were more resistant to water-induced film degradation than the other composites, which required storage in dry air after fabrication. In addition, the HistNH₃CuCl₄·PVDF and HistNH₃CoCl₄·PVDF composites with higher than 5.1 wt. % metallate was not easily peeled and were therefore not chosen for further testing. Attempts to make the Sb-containing composites did not yield easily peeled films in our attempts. PXRD measurements of the films indicated similar diffractions for the Zn-based composite and single crystal, but the Co-based composite had no discernable diffraction besides PVDF and the Cu-based composite did not exhibit diffraction peaks that were similar to those simulated from the crystal. Therefore, it is possible that only the Zn-based composite contained the same metallate structure as was solved by powder X-ray diffraction. The 60×60 μm topography images shown in the insets of FIG. 14 were taken using contact mode at a scan rate of 0.1 Hz. A linear regression analysis was carried out for each measurement. The slope of the voltage vs displacement was taken to obtain the local piezoelectric response (see Supporting Information). FIG. 14 reports the average of the slopes from the 10 different measurements and their standard deviation. To remove the background noise from the measurements, a single measurement was performed on a non-piezoelectric sample (a glass slide) using the same parameters. The measured response of the non-piezoelectric sample was subtracted out of the signals from the polymer composites.

From the PFM measurements on the polymer/metallate composite materials, a similar trend to that observed with single-crystal samples was observed. The HistNH₃ZnCl₄·PVDF composite yielded the highest local piezoelectric response (9.6 ±2.4 pm/V), and the HistNH₃CoCl₄·PVDF yielded a response marginally better response than the reference PVDF film (5.2±1.3 pm/V). The HistNH₃CuCl₄·PVDF composite yielded no improvement over the unmodified PVDF films (4.4±1.2 pm/V), as would be expected for a non-piezoelectric material. The magnitude of the d₃₃ coefficients measured via the Berlincourt method are relatively small compared to the PFM data in Table 2, with values of 0.31 pm/V for HistNH₃ZnCl₄·PVDF, 0.24 pm/V for HistNH₃CoCl₄·PVDF, and 0.09 pm/V for HistNH₃CuCl₄·PVDF. We attribute this due to differences between the Berlincourt piezo-meter, which is a bulk measurement of the metallate composite using upper and lower electrodes, and PFM which is a localized surface measurement that brings a sharp conductive probe into contact with the metallate composite surface. Since PFM is a localized surface measurement, it is less influenced by the polymer phase and therefore tends to exhibit high d₃₃ values, while the Berlincourt piezo-meter operates through thickness mode, and the polarization and measured d₃₃ coefficient is dominated by the continuous polymer phase.

To further confirm that each material was noncentrosymmetric at temperatures similar to those used in PFM measurement, optical second-harmonic generation (SHG) studies were undertook. This is a common way to screen for ferroelectrics because SHG only occurs in materials that lack inversion symmetry. A Zeiss LSM880 upright multiphoton microscope was used with tunable laser power, excitation wavelength and detection wavelength (FIG. 15). Making crystalline powders with specific particle sizes was unsuccessful, so use single crystals was used for obtaining measurements. X-ray quality single crystals were used to verify material identity and then they were sliced with a razor blade to reduce surface irregularities and mounted with an arbitrary crystallographic orientation. the values found were compared to samples of poled PVDF sheets and a large single crystalline potassium dihydrogen phosphate (KDP) sample, both of which are typical piezoelectric reference materials.

Excitation wavelengths from 920-840 nm were scanned and the intensity of λ/2 emission was observed. During the measurement, it was found that each of the Zn-, Co-, and Sb-containing materials produced λ/2 light. For samples with an inversion center, no light was observed. For the two reference materials, the poled PVDF and KDP samples both exhibited a SHG response as well. These data confirm that each of the chlorometallate materials are non-centrosymmetric as grown and as measured by PFM, and not only at the low temperature in which their structures were solved by X-ray crystallography. Because sample thickness, light scattering, absorption, and surface irregularities can play a large role in the magnitude of SHG response measured, these SHG measurement values are not meant to be quantitative. Until powders of each material with specific particle sizes can be reliably formed, then these figures cannot be presented quantitatively.

Herein were synthesized a range of novel organic-inorganic hybrid chlorometallates, including three systems that crystallized in a non-centrosymmetric space group. It has been determined that using different metal B site cations, a range of metallate dimensionality, symmetry, and degree of halide sharing could be accessed when using the histammonium dication. The local piezoelectric response of the three materials has been evaluated using piezoresponse force microscopy, HistNH₃ZnCl₄, HistNH₃CoCl₄, HistNH₃SbCl₅ and it was found that even though they are isostructural, the Zn containing material exhibited a much higher response than the Co containing material. It was verified that each non-centrosymmetric structure retained its low-symmetry structure at room temperature using second harmonic generation spectroscopy. It was then correlated the piezoelectric response difference of the Co and Zn materials to material properties. It was found that the ZnCl₄ containing material exhibited a higher piezoelectric response than CoCl₄ in crystal and composite form, which was attributed to a higher material dipole moment, larger strain tensors, and a much wider bandgap for the ZnCl₄ material than for the CoCl₄ material.

General Considerations: Reagents used as received from the following chemical suppliers: hydrochloric acid and histamine dihydrochloride were purchased from Matrix Scientific and the transition metal chlorides from various sources. Differential scanning calorimetry measurements were acquired on a TA-Instruments TRIOS DSC 2500 from 0 to 200° C.

### Materials and Solutions Preparation

The various histammonium HOIP single crystals were grown by dissolving the transition metal halides M^{x+}Clₓ (M=Fe²⁺, Fe³⁺, Cu²⁺, Co²⁺, Ni²⁺, Zn²⁺, Cd²⁺, Hg²⁺, Sn²⁺, Pb²⁺, Sb³⁺, Bi³⁺) (1.0 mmol) and histammonium dihydrochloride in either 1 mL of hydrochloric acid, 1 mL of water acidified to pH 3 with HCl, 1 mL of 1:1 methanol:water acidified to pH=3, or 1 mL of DMSO in a capped 2 mL vial. In each case, the contents were warmed until dissolution before being left to crystallize. The single-crystal samples were left to grow over the course of several days.

### Metallate Composite Film Preparation

Composite films used for PFM measurements were prepared by dissolving PVDF (10 wt %) and histammoniumMCl₄ (M=Co, Cu, Zn), drop-casting 50 μL of the solution onto a soda lime glass slide, and then annealed in an oven.

### Single-Crystal Characterization

Single-crystal data of the metallates collected on a Bruker D8 Venture κ-axis diffractometer with Mo Kα radiation (0.71073 Å) at 90.0 K. The crystal structures were solved by dual-space methods and refined by full-matrix least-squares using the SHELX programs.

### PFM Characterization

Each sample was mounted onto a polished aluminum puck using carbon sticky tape as a conductive adhesive. Piezoresponse force microscopy (PFM) was performed on all samples as well as a poly(vinylidene difluoride) (PVDF) reference sample at 10 different locations using a Bruker Dimension Icon mounted with a SCM-PIT-V2 platinum-iridium coated conductive tip. To determine the measured displacement of the surface, the tip was calibrated using fused silica to determine the deflection sensitivity. A thermal tuning procedure was also performed to determine the quality factor of the tip (Q=184). A single measurement at a location consisted of measuring the displacement of the sample when applying 0, 2, 4, 6, 8, and 10 V by scanning a 500×167 nm² area at an applied load of 68 nN. At each voltage, the response of the sample was taken to be the average response of the resulting image (averaging over ˜22,000 data points).

Piezoelectric charge coefficients, d₃₃, of all samples with top and bottom electrodes were measured before and after corona poling by the Berlincourt method in a higher precision mode of piezo-meter (PiezoTest PM300, Singapore). For each sample, three measurements in both positive and negative d₃₃ were taken before and after corona poling to evaluate the piezoelectric response of all samples.

### Second Harmonic Generation Measurement

X-ray quality single crystals of Co, Sb, Zn were first sliced with a razorblade and the sliced crystal mounted on a soda lime glass slide with arbitrary crystallographic orientation. The slide was then mounted into a Zeiss LSM880 upright multiphoton microscope and illuminated with light between 840 nm and 920 at a 5× lens objective. The emitted light matching λ/2 nm is detected for all in-focus light and plotted versus the wavelength.

### Experimental Bandgap Determination

Thin films of Cu, Co, Sb, and Zn OIHMs formed by drop casting 1M precursor solutions onto sodalime glass microscope slides, annealing at 100° C. for 5 min and recording the transmission spectra of the thick, polycrystalline film using a Cary5000 UV/Vis/NIR spectrophotometer.

### Computational Details

All DFT calculations were performed using the Vienna ab initio simulation package (VASP) code by utilizing the Perdew-Burke-Ernzerhof (PBE) exchange-correlation functional and the pseudopotentials recommended by VASP. Gaussian smearing with smearing parameter (K_{B}T) of 0.02 eV, energy convergence criterion for the electronic steps equal to 10⁻⁷ eV and the gamma-centered k-meshes with the smallest allowed spacing between k-points equal to 0.03 Å⁻¹ for the Brillouin zone sampling was used in all the calculations. In addition, the calculations of Cu- and Co-based materials were initialized in three different spin configurations (ferromagnetic, ferrimagnetic, and anti-ferromagnetic). However, the subsequent analyses were performed only on the structure corresponding to the minimum energy. For Cu- and Co-based materials we found the minimum energy corresponding to antiferromagnetic and ferromagnetic spin configuration, respectively. We also computed the elastic tensor following the strain-stress relationship and the piezoelectric stress tensor using the linear response theory as described in the literature. Finally, we utilized the Density Derived Electrostatic and Chemical (DDEC6) approach to obtain the net atomic charges and dipole moment on each atom in the calculated structure. LOBSTER package version 4.1.0 was utilized for the chemical-bonding interaction using the “pbevaspfit2015” basis set. The necessary static self-consistent calculations were performed by switching off the symmetry as suggested in the LOBSTER manual.

### Aspects

A 1^{st} aspect of the present disclosure, either alone or in combination with any other aspect herein concerns a halometallate composition comprising a histammonium metal halide.

A 2^{nd} aspect of the present disclosure, either alone or in combination with any other aspect herein concerns the composition of the 1^{st} aspect, wherein the histammonium metal halide comprises the formula HistNH₃MXₙ, wherein M is a metal and X is a halide ion.

A 3^{rd} aspect of the present disclosure, either alone or in combination with any other aspect herein concerns the composition of the 1^{st} or 2^{nd} aspect, wherein the metal is selected from iron iron (Fe), copper (Cu), cobalt (Co), nickel (Ni), zinc (Zn), antimony (Sb), tin (Sn), cadmium (Cd), mercury (Hg), lead (Pb), bismuth (Bi), scandium (Sc), titanium (Ti), vanadium (V), chromium (Cr), manganese (Mn), gallium (Ga), germanium (Ge), and arsenic (As), zirconium (Zr), molybdenum (Mo), ruthenium (Ru), rhodium (Rh), palladium (Pd), silver (Ag), and gold (Au).

A 4^{th} aspect of the present disclosure, either alone or in combination with any other aspect herein concerns the composition of the 2^{nd} or 3^{rd} aspect, wherein X is selected from chlorine (Cl), fluorine (F), bromine (Br), Iodine (I), and astatine (At).

A 5^{th} aspect of the present disclosure, either alone or in combination with any other aspect herein concerns the composition of the 1^{st} through 4^{th} aspects, wherein the histammonium metal halide is HistNH₃CoCl₄.

A 6^{th} aspect of the present disclosure, either alone or in combination with any other aspect herein concerns the composition of the 1^{st} through 4^{th} aspects, wherein the histammonium metal halide is HistNH₃CuCl₄.

A 7^{th} aspect of the present disclosure, either alone or in combination with any other aspect herein concerns the composition of the 1^{st} through 4^{th} aspects, wherein the histammonium metal halide is HistNH₃ZnCl₄.

An 8^{th} aspect of the present disclosure, either alone or in combination with any other aspect herein concerns the composition of the 1^{st} through 4^{th} aspects, wherein the histammonium metal halide is HistNH₃NiCl₄.

A 9^{th} aspect of the present disclosure, either alone or in combination with any other aspect herein concerns the composition of the 1^{st} through 4^{th} aspects, wherein the histammonium metal halide is HistNH₃CdCl₄.

A 10^{th} aspect of the present disclosure, either alone or in combination with any other aspect herein concerns the composition of the 1^{st} through 4^{th} aspects, wherein the histammonium metal halide is HistNH₃HgCl₄.

An 11^{th} aspect of the present disclosure, either alone or in combination with any other aspect herein concerns the composition of the 1^{st} through 4^{th} aspects, wherein the histammonium metal halide is HistNH₃SnCl₄.

A 12^{th} aspect of the present disclosure, either alone or in combination with any other aspect herein concerns the composition of the 1^{st} through 4^{th} aspects, wherein the histammonium metal halide is HistNH₃PbCl₄.

A 13^{th} aspect of the present disclosure, either alone or in combination with any other aspect herein concerns the composition of the 1^{st} through 4^{th} aspects, wherein the histammonium metal halide is HistNH₃SbCl₅.

A 14^{th} aspect of the present disclosure, either alone or in combination with any other aspect herein concerns the composition of the 1^{st} through 4^{th} aspects, wherein the histammonium metal halide is (HistNH₃)₃(BiCl₆)₂.

A 15^{th} aspect of the present disclosure, either alone or in combination with any other aspect herein concerns the composition of the 1^{st} through 4^{th} aspects, wherein the histammonium metal halide is HistNH₃PbBr₄.

A 16^{th} aspect of the present disclosure, either alone or in combination with any other aspect herein concerns the composition of the 1^{st} through 4^{th} aspects, wherein the histammonium metal halide is HistNH₃HgBr₄.

A 17^{th} aspect of the present disclosure, either alone or in combination with any other aspect herein concerns the composition of the 1^{st} through 4^{th} aspects, wherein the histammonium metal halide is HistNH₃SnBr₄.

An 18^{th} aspect of the present disclosure, either alone or in combination with any other aspect herein concerns the composition of the 1^{st} through 4^{th} aspects, wherein the histammonium metal halide is HistNH₃MnCl₄.

A 19^{th} aspect of the present disclosure, either alone or in combination with any other aspect herein concerns the composition of the 1^{st} through 4^{th} aspects, wherein the histammonium metal halide is HistNH₃PdCl₄.

A 20^{th} aspect of the present disclosure, either alone or in combination with any other aspect herein concerns the composition of the 1^{st} through 4^{th} aspects, wherein the histammonium metal halide is HistNH₃PdBr₄.

A 21^{st} aspect of the present disclosure, either alone or in combination with any other aspect herein concerns the composition of the 1^{st} through 4^{th} aspects, wherein the histammonium metal halide is HistNH₃RuCl₄.

A 22^{nd} aspect of the present disclosure, either alone or in combination with any other aspect herein concerns the composition of the 1^{st} through 4^{th} aspects, wherein the histammonium metal halide is HistNH₃RuBr₄.

A 23^{rd} aspect of the present disclosure, either alone or in combination with any other aspect herein concerns the composition of the 1^{st} through 4^{th} aspects, wherein the histammonium metal halide is HistNH₃ZnBr₄.

A 24^{th} aspect of the present disclosure, either alone or in combination with any other aspect herein concerns the composition of the 1^{st} through 4^{th} aspects, wherein the histammonium metal halide is HistNH₃MnBr₄.

A 25^{th} aspect of the present disclosure, either alone or in combination with any other aspect herein concerns the composition of the 1^{st} through 4^{th} aspects, wherein the histammonium metal halide is HistNH₃CoBr₄.

A 26^{th} aspect of the present disclosure, either alone or in combination with any other aspect herein concerns the composition of the 1^{st} through 4^{th} aspects, wherein the histammonium metal halide is HistNH₃NiBr₄.

A 27^{th} aspect of the present disclosure, either alone or in combination with any other aspect herein concerns the composition of the 1^{st} through 4^{th} aspects, wherein the histammonium metal halide is HistNH₃CuBr₄.

A 28^{th} aspect of the present disclosure, either alone or in combination with any other aspect herein concerns the composition of the 1^{st} through 4^{th} aspects, wherein the histammonium metal halide is (HistNH₃)₃(BiBr₆)₂.

A 29^{th} aspect of the present disclosure, either alone or in combination with any other aspect herein concerns the composition of the 1^{st} through 4^{th} aspects, wherein the histammonium metal halide is HistNH₃RuI₄.

A 30^{th} aspect of the present disclosure, either alone or in combination with any other aspect herein concerns the composition of the 1^{st} through 4^{th} aspects, wherein the histammonium metal halide is HistNH₃ZnI₄.

A 31^{st} aspect of the present disclosure, either alone or in combination with any other aspect herein concerns the composition of the 1^{st} through 4^{th} aspects, wherein the histammonium metal halide is HistNH₃MnI₄.

A 32^{nd} aspect of the present disclosure, either alone or in combination with any other aspect herein concerns the composition of the 1^{st} through 4^{th} aspects, wherein the histammonium metal halide is HistNH₃CoI₄.

A 33^{rd} aspect of the present disclosure, either alone or in combination with any other aspect herein concerns the composition of the 1^{st} through 4^{th} aspects, wherein the histammonium metal halide is HistNH₃NiI₄.

A 34^{th} aspect of the present disclosure, either alone or in combination with any other aspect herein concerns the composition of the 1^{st} through 4^{th} aspects,, wherein the histammonium metal halide is HistNH₃CuI₄.

A 35^{th} aspect of the present disclosure, either alone or in combination with any other aspect herein concerns the composition of the 1^{st} through 4^{th} aspects, wherein the histammonium metal halide is (HistNH₃)₃(BiI₆)₂

A 36^{th} aspect of the present disclosure, either alone or in combination with any other aspect herein concerns the composition of the 1^{st} through 4^{th} aspects, wherein the histammonium metal halide is HistNH₃PdI₄.

A 37^{th} aspect of the present disclosure, either alone or in combination with any other aspect herein concerns the composition of the 1^{st} through 4^{th} aspects, wherein the histammonium metal halide is HistNH₃PbI₄.

A 38^{th} aspect of the present disclosure, either alone or in combination with any other aspect herein concerns the composition of the 1^{st} through 4^{th} aspects, wherein the histammonium metal halide is HistNH₃HgI₄.

A 39^{th} aspect of the present disclosure, either alone or in combination with any other aspect herein concerns the composition of the 1^{st} through 4^{th} aspects, wherein the histammonium metal halide is HistNH₃SnI₄.

A 40^{th} aspect of the present disclosure, either alone or in combination with any other aspect herein concerns the composition of the 1^{st} through 4^{th} aspects, wherein the histammonium metal halide is HistNH₃SbI₅.

A 41^{st} aspect of the present disclosure, either alone or in combination with any other aspect herein concerns the composition of the 1^{st} through 4^{th} aspects, wherein the histammonium metal halide is HistNH₃CdI₄.

A 42^{nd} aspect of the present disclosure, either alone or in combination with any other aspect herein concerns the composition of the 1^{st} through 41^{st} aspects, further comprising a non-reactive polymer composite.

A 43^{rd} aspect of the present disclosure, either alone or in combination with any other aspect herein concerns the composition of the 42^{nd} aspect, wherein the polymer composite is selected from polyvinylidene fluoride (PVDF), polydimethylsiloxane (PDMS), polydiethylsiloxane (PDES), polymethylphenylsiloxane (PMPS), polyvinylidene chloride (PVDC), cellulose acetate, ethylene vinyl acetate, high density polyethylene, polycarbonate, polyester (PET or PEN), polylactic acid, polyurethane, polyvinyl alcohol, polyvinyl butyral, polyvinyl chloride, polyvinyl fluoride, polypropylene, polystyrene, low density polyethylene, cellulose, polytetrafluoroethylene (PTFE), polyimide, melamine, nylon, or combinations thereof.

A 44^{th} aspect of the present disclosure, either alone or in combination with any other aspect herein concerns the composition of the 42^{nd} or 43^{rd} aspects, wherein the polymer composite is PVDF or PDMS.

A 45^{th} aspect of the present disclosure, either alone or in combination with any other aspect herein concerns a piezoelectric device comprising the halometallate composition of any of the 1^{st} through 44^{th} aspects.

A 46^{th} aspect of the present disclosure, either alone or in combination with any other aspect herein concerns the piezoelectric device of the 45^{th} aspect, further comprising a first electrode in contact with the halometallate composition.

A 47^{th} aspect of the present disclosure, either alone or in combination with any other aspect herein concerns the piezoelectric device of the 45^{th} or 46^{th} aspects, further comprising a substrate.

A 48^{th} aspect of the present disclosure, either alone or in combination with any other aspect herein concerns the piezoelectric device of the 47^{th} aspect, wherein the substrate is a glass or polymer.

A 49^{th} aspect of the present disclosure, either alone or in combination with any other aspect herein concerns the piezoelectric device of the 47^{th} or 48^{th} aspect, further comprising a second electrode.

A 50^{th} aspect of the present disclosure, either alone or in combination with any other aspect herein concerns the piezoelectric device of the 9^{th} aspect, wherein the second electrode is in contact with the halometallate composition.

A 51^{st} aspect of the present disclosure, either alone or in combination with any other aspect herein concerns the piezoelectric device of the 50^{th} aspect, wherein a first surface of the halometallate composition is layered at least in part on the first electrode and a second surface of the halometaalate composition is layered at least in part on the second electrode.

A 52^{nd} aspect of the present disclosure, either alone or in combination with any other aspect herein concerns the piezoelectric device of the 51^{st} aspect, wherein the substrate is also layered on the first electrode.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. A halometallate composition comprising a histammonium metal halide.

2. The composition of claim 1, wherein the histammonium metal halide comprises the formula HistNH₃MX, wherein M is a metal and X is a halide ion.

3. The composition of claim 1, wherein the metal is selected from iron (Fe), copper (Cu), cobalt (Co), nickel (Ni), zinc (Zn), antimony (Sb), tin (Sn), cadmium (Cd), mercury (Hg), lead (Pb), bismuth (Bi), scandium (Sc), titanium (Ti), vanadium (V), chromium (Cr), manganese (Mn), gallium (Ga), germanium (Ge), arsenic (As) zirconium (Zr), molybdenum (Mo), ruthenium (Ru), rhodium (Rh), palladium (Pd), silver (Ag), and gold (Au).

4. The composition of claim 2, wherein X is selected from chlorine (Cl), fluorine (F), bromine (Br), Iodine (I), and astatine (At).

5. The composition of claim 1, wherein the histammonium metal halide is selected from HistNH₃CoCl₄, HistNH₃CuCl₄, HistNH₃ZnCl₄, HistNH₃NiCl₄, HistNH₃CdCl₄, HistNH₃HgCl₄, HistNH₃SnCl₄, HistNH₃PbCl₄, HistNH₃SbCl₅, (HistNH₃)₃(BiCl₆)₂, HistNH₃PbBr₄, HistNH₃HgBr₄, HistNH₃SnBr₄, HistNH₃MnCl₄, HistNH₃PdCl₄, HistNH₃PdBr₄, HistNH₃RuCl₄, HistNH₃RuBr₄, HistNH₃ZnBr₄, HistNH₃MnBr₄, HistNH₃CoBr₄, HistNH₃NiBr₄, HistNH₃CuBr₄, HistNH₃(BiBr₆)₂, HistNH₃RuI₄, HistNH₃ZnI₄, HistNH₃MnI₄, HistNH₃CoI₄, HistNH₃NiI₄, HistNH₃CuI₄, HistNH₃(BiI₆)₂, HistNH₃PdI₄, HistNH₃PbI₄, HistNH₃HgI₄, HistNH₃SnI₄, HistNH₃SbI₅, and HistNH₃CdI₄,

6. The composition of claim 5, wherein the histammonium metal halid is HistNH₃CuCl₄.

7. The composition of claim 5, wherein the histammonium metal halide is HistNH₃ZnCl₄.

8. The composition of claim 5, wherein the histammonium metal halide is HistNH₃CoCl₄.

9-41. (canceled)

42. The composition of claim 1, further comprising a non-reactive polymer composite.

43. The composition of claim 42, wherein the polymer composite is selected from polyvinylidene fluoride (PVDF), polydimethylsiloxane (PDMS), polydiethylsiloxane (PDES), polymethylphenylsiloxane (PMPS), polyvinylidene chloride (PVDC), cellulose acetate, ethylene vinyl acetate, high density polyethylene, polycarbonate, polyester (PET or PEN), polylactic acid, polyurethane, polyvinyl alcohol, polyvinyl butyral, polyvinyl chloride, polyvinyl fluoride, polypropylene, polystyrene, low density polyethylene, cellulose, polytetrafluoroethylene (PTFE), polyimide, melamine, nylon, or combinations thereof.

44. The composition of claim 42, wherein the polymer composite is PVDF or PDMS.

45. A piezoelectric device comprising the halometallate composition of claim 1.

46. The piezoelectric device of claim 45, further comprising a first electrode in contact with the halometallate composition.

47. The piezoelectric device of claim 45, further comprising a substrate.

48. The piezoelectric device of claim 47, wherein the substrate is a glass or polymer.

49. The piezoelectric device of claim 47, further comprising a second electrode.

50. The piezoelectric device of claim 59, wherein the second electrode is in contact with the halometallate composition.

51. The piezoelectric device 50, wherein a first surface of the halometallate composition is layered at least in part on the first electrode and a second surface of the halometaalate composition is layered at least in part on the second electrode.

52. The piezoelectric device of claim 51, wherein the substrate is also layered on the first electrode.
